(19) United States
(12) Patent Application Publication
(10) Pub. No.: US 2020/0138744 A1
(43) Pub. Date: May 7, 2020

(54) METHODS OF TREATING PANCREATIC CANCER USING COENZYME Q10

(71) Applicant: Berg LLC, Framingham, MA (US)

(72) Inventors: Rangaprasad Sarangarajan, Boylston, MA (US); Niven Rajin Narain, Cambridge, MA (US)

(21) Appl. No.: 16/653,787

(22) Filed: Oct. 15, 2019

Related U.S. Application Data
(60) Provisional application No. 62/745,957, filed on Oct. 15, 2018.

(51) Int. Cl. A61K 31/122; A61P 35/04; A61K 9/00; A61K 31/7068

(52) U.S. Cl. CPC A61K 31/122; A61K 31/7068; A61K 9/0019; A61P 35/04

(57) ABSTRACT

The disclosure provides methods of treating pancreatic cancer in a population of subjects comprising administration of Coenzyme Q10. Administration of Coenzyme Q10 to the subjects increases the number of subjects in the population that exhibit stable disease. In addition, administration of Coenzyme Q10 to the subjects increases progression free survival, overall survival, and time to progression in the population of pancreatic cancer patients.

### RELATED APPLICATIONS

This application claims priority to U.S. Provisional Patent Application No. 62/745,957 filed on Oct. 15, 2018, the contents of which are incorporated herein in their entirety.

### BACKGROUND OF THE INVENTION

In 2018, it is estimated that 55,440 adults (29,200 men and 26,240 women) in the United States will be diagnosed with pancreatic cancer. Of these, approximately 44,330 deaths will occur because of the disease. Pancreatic cancer is the third leading cause of cancer death with a 5-year survival rate of approximately 8.5%. Accordingly, a need exists for improved pancreatic cancer treatments.

### SUMMARY OF THE INVENTION

In certain aspects, the disclosure relates to a method of treating pancreatic cancer in a population of subjects in need thereof, the method comprising administering to each subject of the population a composition comprising Coenzyme Q10, wherein at least 10%, 20%, 30%, 40%, 50% or 53% of the subjects in the population exhibit stable disease as a result of administration of the Coenzyme Q10 to the subjects, thereby treating the pancreatic cancer in the population of subjects. In certain embodiments, at least 5%, 10%, 15%, 20% or 23% of the subjects in the population exhibit stable disease for at least four months as a result of administration of the Coenzyme Q10 to the subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median progression free survival of at least 50, 60, 70, 80, 90, 100, 110, or 118 days as a result of administration of the Coenzyme Q10 to the subjects, thereby treating the pancreatic cancer in the population of subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median overall survival of at least 50, 60, 70, 80, 90, 100, 110, 120, 125, 150, 175, 200 or 218 days as a result of administration of the Coenzyme Q10 to the subjects, thereby treating the pancreatic cancer in the population of subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median time to progression of at least 10, 20, 30, 40, 50, 60, 65, 68, 70, 80, 90, 100, 110, 120 or 121 days as a result of administration of the Coenzyme Q10 to the subjects, thereby treating the pancreatic cancer in the population of subjects.

In certain embodiments, the composition comprising Coenzyme Q10 is formulated for intravenous administration. In certain embodiments, the composition comprising Coenzyme Q10 is administered by continuous intravenous infusion. In certain embodiments, the composition comprising Coenzyme Q10 is administered by continuous intravenous infusion for at least 24, 48, 72, 96, 120 or 144 hours. In certain embodiments, the composition comprising Coenzyme Q10 is administered by continuous intravenous infusion for at least 144 hours. In certain embodiments, the continuous intravenous infusion is administered to the subject for at least 30 days over a ten week period. In certain embodiments, the coenzyme Q10 is administered at a dose of at least 20, 30, 40, 50, 60, 70, 80, 90, 100 or 110 mg/kg/72 hours. In certain embodiments, he coenzyme Q10 is administered at a dose of at least 110 mg/kg/72 hours.

In certain embodiments, the subject is a human. In certain embodiments, the pancreatic cancer is metastatic. In certain embodiments, each subject in the population has had at least one prior therapy for the pancreatic cancer. In certain embodiments, each subject in the population has had no more than 2 prior therapies for the pancreatic cancer. In certain embodiments, each subject in the population has failed treatment for the pancreatic cancer with at least one prior therapy.

In certain embodiments, the composition comprising Coenzyme Q10 is administered with one or more additional agents. In certain embodiments, the additional agent is an anti-cancer agent. In certain embodiments, the additional agent is a chemotherapeutic agent. In certain embodiments, the chemotherapeutic agent is selected from the group consisting of cisplatin, gemcitabine (gemzar), mitomycin, 5-fluorouracil (5-FU), paclitaxel (taxol), and irinotecan. In certain embodiments, the chemotherapeutic agent is gemcitabine. In certain embodiments, the gemcitabine is administered intravenously at a dose of at least 1000 mg/m². In certain embodiments, the method further comprises evaluating each of the subjects by RECIST criteria. In certain embodiments, each of the subjects is an evaluable subject.

### BRIEF DESCRIPTION OF THE DRAWINGS

FIG. 1 shows the time to progression in pancreatic cancer patients treated with Coenzyme Q10 monotherapy or combination therapy.

FIG. 2 shows overall survival of pancreatic cancer patients treated with Coenzyme Q10 monotherapy or combination therapy.

FIG. 3 shows the time to progression in pancreatic cancer patients treated with Coenzyme Q10 and gemcitabine.

FIG. 4 shows progression free survival days in pancreatic cancer patients treated with Coenzyme Q10 and gemcitabine.

FIG. 5 shows overall survival days in pancreatic cancer patients treated with Coenzyme Q10 and gemcitabine.

### DETAILED DESCRIPTION

### I. Definitions

As used herein the term “evaluable subject” refers to a subject having pancreatic cancer that has received Coenzyme Q10 treatment by continuous intravenous infusion for at least 30 days over a ten week period, has received at least one dose of gemcitabine, and has had a RECIST 1.1 evaluation.

Specific criteria for the staging of pancreatic cancer are based on tumor size, histological characteristics, tumor markers, and other criteria known by those of skill in the art. Generally, pancreatic cancer stages can be described as follows:

Stage 0—Carcinoma in situ

Stage I, Stage II, and Stage III—Higher numbers indicate more extensive disease: Larger tumor size and/or spread of the cancer beyond the organ in which it first developed to nearby lymph nodes and/or tissues or organs adjacent to the location of the primary tumor

Stage IV—The cancer has spread to distant tissues or organs

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the quality of life (QOL) as judged by a qualified individual, e.g., a treating physician, e.g., using accepted assessment tools of pain and QOL is not considered to be a “treatment” of the cancer.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al. (New response evaluation criteria in solid tumors: Revised RECIST guideline (version 1.1) Eur. J. Cancer. 45:228-247, 2009), the entire contents of which are incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Disease (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for Partial Response (PR) nor sufficient increase to qualify for Progressive Disease (PD), taking as a reference the smallest sum diameters while on study.

RECIST 1.1 criteria also consider non-target lesions which are defined as lesions that may be measurable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “chemotherapeutic regimen” is a clinically accepted dosing protocol for the treatment of cancer that includes administration of one or more chemotherapeutic agents to a subject in specific amounts on a specific schedule. In certain embodiments, the chemotherapeutic agent can be an agent in clinical trials.

Chemotherapeutic regimens can include administration of a drug on a predetermined “cycle” including intervals of dosing and not dosing with one or more agents for the treatment of cancer. For example, an agent can be administered one or more times per week for three consecutive weeks followed by a week of no agent administered to provide a four week cycle. The cycle can be repeated so that the subject would be subjected to three treatment weeks, one no treatment week, three treatment weeks, one no treatment week, etc., for the desired number of cycles. In certain embodiments, treatment of efficacy and laboratory values (e.g., liver enzymes, blood count, kidney function) are assessed at the end of each cycle or every other cycle.

A “subject who has failed a chemotherapeutic regimen” is a subject with cancer (e.g. pancreatic cancer) that does not respond, or ceases to respond to treatment with a chemotherapeutic regimen per RECIST 1.1 criteria (see, Eisenhauer et al., 2009 and as discussed above), i.e., does not achieve at least stable disease (i.e., stable disease, partial response, or complete response) in the target lesion; or does not achieve at least non-CR/non-PD (i.e., non-CR/non-PD or complete response) of non-target lesions, either during or after completion of the chemotherapeutic regimen, either alone or in conjunction with surgery and/or radiation therapy which, when possible, are often clinically indicated in conjunction with chemotherapy. A failed chemotherapeutic regime results in, e.g., tumor growth, increased tumor burden, and/or tumor metastasis. In some embodiments, failed chemotherapeutic regimen as used herein includes a treatment regimen that was terminated due to a dose limiting toxicity, e.g., a grade III or a grade IV toxicity that cannot be resolved to allow continuation or resumption of treatment with the chemotherapeutic agent or regimen that caused the toxicity. In some embodiments, a “failed chemotherapeutic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. In some embodiments, a failed chemotherapeutic regimen includes a treatment regimen that results in progressive disease of at least one target lesion during treatment with the chemotherapeutic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed chemotherapeutic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen. For example, a subject who suffered from a pediatric cancer may develop cancer later in life after being cured of the pediatric cancer. In such a subject, the chemotherapeutic regimen to treat the pediatric cancer is considered to have been successful.

A “refractory cancer” is a malignancy for which surgery is ineffective, which is either initially unresponsive to chemo- or radiation therapy, or which becomes unresponsive to chemo- or radiation therapy over time.

A “therapeutically effective amount” is that amount sufficient to treat a disease in a subject. A therapeutically effective amount can be administered in one or more administrations.

The terms “administer”, “administering” or “administration” include any method of delivery of a pharmaceutical composition or agent into a subject's system or to a particular region in or on a subject. In a preferred embodiment, the formulations of the invention may be administered by intravenous injection or intravenous infusion. In still more preferred embodiments, the formulation of the invention can be administered by continuous infusion.

As used herein, “continuous infusion” refers to administration of a dose of the formulation continuously for at least 24 hours. Continuous administration is typically facilitated by use of a pump, either an implantable or external pump. A formulation can be administered by continuous infusion in multiple, separated doses, with a break of one or more days between continuous infusion doses.

It is understood that continuous infusion can include short interruptions of administration, for example, to change the reservoir of coenzyme Q10 being administered. For example, two 48 hour continuous infusions administered sequentially or four 24 hour continuous infusions and the like, administered without a significant pause by design (less than 4 hours, preferably less than 2 hours, preferably less than one hour, preferably about 30 minutes) between the end of one infusion and the start of the next is considered to be the same as one 96 hour continuous administration. Similarly, two 72 hour continuous infusions administered sequentially without a significant pause (e.g. less than 4 hours, preferably less than 2 hours, preferably less than one hour, preferably about 30 minutes) between the end of one infusion and the start of the next is considered to be the same as one 144 hour (6 day) continuous infusion. In certain embodiments, the subject treated with a 96 hour continuous infusion of coenzyme Q10 or a 144 hour continuous infusion of coenzyme Q10 has pancreatic cancer.

An 18 day continuous infusion will similarly likely include short interruptions during the dosing and will be considered to be a continuous infusion as long as the dose is administered without significant pause by design (less than 4 hours, preferably less than 2 hours, preferably less than one hour, preferably about 30 minutes) between the end of one infusion and the start of the next. In certain embodiments, the subject treated with an 18 day continuous infusion of coenzyme Q10 has pancreatic cancer. It is understood that one day of a multi-day continuous infusion is one of a series of sequential 24 hour periods during the continuous infusion that does not necessarily, or even typically, correspond to a calendar day. For example, day 1 of a continuous infusion includes hours 1-24 of the continuous infusion, day 2 of a continuous infusion includes hours 25-48 of the continuous infusion, day 3 of a continuous infusion includes hours 49-72 of the continuous infusion, etc.

Continuous administration is typically facilitated by the use of a pump. Continuous administration can include administration at a single rate. Continuous administration can include administration at a more than one rate (e.g., two rates, three rates). Continuous administration can include a loading dose at a higher rate followed by a lower dose rate for the remainder of the dose. Continuous infusion is carried out without including any significant interruptions of dosing by design. As used herein, interruptions to assess vital signs and/or perform laboratory assessments to ensure the safety of the patients and that no unacceptable adverse event have occurred are not considered to be significant interruptions. Interruptions resulting from equipment failure, e.g., pump failure, are not interruptions by design.

As used herein, continuous administration does not include intravenous administration of two or more doses wherein the end of the first dose and the start of the second dose are separated from each other by at least four hours, preferably at least eight, twelve, or twenty four hours by design. When two intravenous administrations are separated by at least four hours by design, they are separate doses.

In certain embodiments, a continuous infusion is designed for administration at a single rate. In certain embodiments, a continuous infusion is designed for administration at a higher rate at the beginning of the infusion to provide a loading dose. For example, the dose for administration during the first 24 hours of the continuous infusion can be administered at two rates such that at least 5% of the dose is administered in the first hour, and the remainder of the dose (95% of the dose) for the first 24 hours is administered in the remaining 23 hours. In certain embodiments, the dose for administration during the first 24 hours of the continuous infusion can be administered at two rates such that at least 6% of the dose is administered in the first hour, and the remainder of the dose (94% of the dose) for the first 24 hours is administered in the remaining 23 hours. For example, the dose for administration during the first 24 hours of the continuous infusion can be administered at two rates such that at least 7% of the dose is administered in the first hour, and the remainder of the dose (95% of the dose) for the first 24 hours is administered in the remaining 23 hours. For example, the dose for administration during the first 24 hours of the continuous infusion can be administered at two rates such that at least 8% of the dose is administered in the first hour, and the remainder of the dose (92% of the dose) for the first 24 hours is administered in the remaining 23 hours. For example, the dose for administration during the first 24 hours of the continuous infusion can be administered at two rates such that at least 9% of the dose is administered in the first hour, and the remainder of the dose (91% of the dose) for the first 24 hours is administered in the remaining 23 hours. For example, the dose for administration during the first 24 hours of the continuous infusion can be administered at two rates such that at least 10% of the dose is administered in the first hour, and the remainder of the dose (90% of the dose) for the first 24 hours is administered in the remaining 23 hours. For example, the dose for administration during the first 24 hours of the continuous infusion can be administered at two rates such that at least 11% of the dose is administered in the first hour, and the remainder of the dose (89% of the dose) for the first 24 hours is administered in the remaining 23 hours. For example, the dose for administration during the first 24 hours of the continuous infusion can be administered at two rates such that at least 12% of the dose is administered in the first hour, and the remainder of the dose (88% of the dose) for the first 24 hours is administered in the remaining 23 hours. For example, the dose for administration during the first 24 hours of the continuous infusion can be administered at two rates such that at least 13% of the dose is administered in the first hour, and the remainder of the dose (87% of the dose) for the first 24 hours is administered in the remaining 23 hours. For example, the dose for administration during the first 24 hours of the continuous infusion can be administered at two rates such that at least 14% of the dose is administered in the first hour, and the remainder of the dose (86% of the dose) for the first 24 hours is administered in the remaining 23 hours. For example, the dose for administration during the first 24 hours of the continuous infusion can be administered at two rates such that at least 15% of the dose is administered in the first hour, and the remainder of the dose (85% of the dose) for the first 24 hours is administered in the remaining 23 hours. In certain embodiments, at least 16%, 17%, 18%, 19%, 20%, 21%, 22%, 23%, 24% or 25% of the dose is administered in the first hour, with the remainder being administered in the subsequent 23 hours.

In certain embodiments about 5%, about 6%, about 7%, about 8%, about 8.1%, about 8.2%, about 8.3%, about 8.4%, about 8.5%, about 8.6%, about 8.7%, about 8.8%, about 8.9%, about 9%, about 10%, about 11%, about 12%, about 13%, about 14%, about 15%, about 16%, about 17%, about 18%, about 19%, about 20% about 21% about 22%, about 23%, about 24%, or about 25% of the dose is administered in the first hour, and the remainder of the dose for the first 24 hours, for example, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, or about 75% is administered in the subsequent 23 hours. Any of these values may be used to define a range for the percentage of the dose that is administered in the first hour and the percentage of the dose administered in the subsequent 23 hours. For example, the percentage of the dose that is administered in the first hour may range from 5% to 25%, 5% to 20%, 5% to 15%, 5% to 10%, 10% to 25%, 10% to 20%, 10% to 15%, 15% to 25%, 15% to 20%, 20% to 25%, 8% to 25%, 8% to 15%, 6% to 9%, or 7% to 9%. The remaining dose is administered in the subsequent 23 hours. For example, the percentage of the dose that is administered in the subsequent 23 hours may range from 75% to 95%, 75% to 90%, 75% to 85%, 75% to 80%, 80% to 95%, 80% to 90%, 80% to 85%, 85% to 95%, 85% to 90%, 90% to 95%, 91% to 94%, 91% to 93%, from 75% to 92%, or from 85% to 92%.

In certain embodiments, the dose for administration during the first 48 hours of the continuous infusion can be administered at two rates such that at least 5% of the dose is administered in the first hour, and the remainder of the dose (95% of the dose) for the first 48 hours is administered in the remaining 47 hours. In certain embodiments, the dose for administration during the first 48 hours of the continuous infusion can be administered at two rates such that at least 6% of the dose is administered in the first hour, and the remainder of the dose (94% of the dose) for the first 48 hours is administered in the remaining 47 hours. For example, the dose for administration during the first 48 hours of the continuous infusion can be administered at two rates such that at least 7% of the dose is administered in the first hour, and the remainder of the dose (95% of the dose) for the first 48 hours is administered in the remaining 47 hours. For example, the dose for administration during the first 48 hours of the continuous infusion can be administered at two rates such that at least 8% of the dose is administered in the first hour, and the remainder of the dose (92% of the dose) for the first 48 hours is administered in the remaining 23 hours. For example, the dose for administration during the first 48 hours of the continuous infusion can be administered at two rates such that at least 9% of the dose is administered in the first hour, and the remainder of the dose (91% of the dose) for the first 48 hours is administered in the remaining 47 hours. For example, the dose for administration during the first 48 hours of the continuous infusion can be administered at two rates such that at least 10% of the dose is administered in the first hour, and the remainder of the dose (90% of the dose) for the first 48 hours is administered in the remaining 47 hours. For example, the dose for administration during the first 48 hours of the continuous infusion can be administered at two rates such that at least 11% of the dose is administered in the first hour, and the remainder of the dose (89% of the dose) for the first 48 hours is administered in the remaining 47 hours. For example, the dose for administration during the first 48 hours of the continuous infusion can be administered at two rates such that at least 12% of the dose is administered in the first hour, and the remainder of the dose (88% of the dose) for the first 48 hours is administered in the remaining 47 hours. For example, the dose for administration during the first 48 hours of the continuous infusion can be administered at two rates such that at least 13% of the dose is administered in the first hour, and the remainder of the dose (87% of the dose) for the first 48 hours is administered in the remaining 47 hours. For example, the dose for administration during the first 48 hours of the continuous infusion can be administered at two rates such that at least 14% of the dose is administered in the first hour, and the remainder of the dose (86% of the dose) for the first 48 hours is administered in the remaining 47 hours. For example, the dose for administration during the first 48 hours of the continuous infusion can be administered at two rates such that at least 15% of the dose is administered in the first hour, and the remainder of the dose (85% of the dose) for the first 48 hours is administered in the remaining 47 hours. In certain embodiments, at least 16%, 17%, 18%, 19%, 20%, 21%, 22%, 23%, 24% or 25% of the dose is administered in the first hour, with the remainder being administered in the subsequent 47 hours.

In certain embodiments about 5%, about 6%, about 7%, about 8%, about 8.1%, about 8.2%, about 8.3%, about 8.4%, about 8.5%, about 8.6%, about 8.7%, about 8.8%, about 8.9% about 9%, about 10%, about 11%, about 12%, about 13%, about 14%, about 15%, about 16%, about 17%, about 18%, about 19%, about 20% about 21% about 22%, about 23%, about 24%, or about 25% of the dose is administered in the first hour, and the remainder of the dose for the first 48 hours, for example, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, or about 75% is administered in the subsequent 47 hours. Any of these values may be used to define a range for the percentage of the dose that is administered in the first hour and the percentage of the dose administered in the subsequent 47 hours. For example, the percentage of the dose that is administered in the first hour may range from 5% to 25%, 5% to 20%, 5% to 15%, 5% to 10%, 10% to 25%, 10% to 20%, 10% to 15%, 15% to 25%, 15% to 20%, 20% to 25%, 8% to 25%, 8% to 15%, 6% to 9%, or 7% to 9%. The remaining dose is administered in the subsequent 47 hours. For example, the percentage of the dose that is administered in the subsequent 47 hours may range from 75% to 95%, 75% to 90%, 75% to 85%, 75% to 80%, 80% to 95%, 80% to 90%, 80% to 85%, 85% to 95%, 85% to 90%, 90% to 95%, 91% to 94%, 91% to 93%, from 75% to 92%, or from 85% to 92%.

In certain embodiments, the dose for administration during the first 72 hours of the continuous infusion can be administered at two rates such that at least 5% of the dose is administered in the first hour, and the remainder of the dose (95% of the dose) for the first 72 hours is administered in the remaining 71 hours. In certain embodiments, the dose for administration during the first 72 hours of the continuous infusion can be administered at two rates such that at least 6% of the dose is administered in the first hour, and the remainder of the dose (94% of the dose) for the first 72 hours is administered in the remaining 71 hours. For example, the dose for administration during the first 72 hours of the continuous infusion can be administered at two rates such that at least 7% of the dose is administered in the first hour, and the remainder of the dose (95% of the dose) for the first 72 hours is administered in the remaining 71 hours. For example, the dose for administration during the first 72 hours of the continuous infusion can be administered at two rates such that at least 8% of the dose is administered in the first hour, and the remainder of the dose (92% of the dose) for the first 72 hours is administered in the remaining 71 hours. For example, the dose for administration during the first 72 hours of the continuous infusion can be administered at two rates such that at least 9% of the dose is administered in the first hour, and the remainder of the dose (91% of the dose) for the first 72 hours is administered in the remaining 71 hours. For example, the dose for administration during the first 72 hours of the continuous infusion can be administered at two rates such that at least 10% of the dose is administered in the first hour, and the remainder of the dose (90% of the dose) for the first 72 hours is administered in the remaining 71 hours. For example, the dose for administration during the first 72 hours of the continuous infusion can be administered at two rates such that at least 11% of the dose is administered in the first hour, and the remainder of the dose (89% of the dose) for the first 72 hours is administered in the remaining 71 hours. For example, the dose for administration during the first 72 hours of the continuous infusion can be administered at two rates such that at least 12% of the dose is administered in the first hour, and the remainder of the dose (88% of the dose) for the first 72 hours is administered in the remaining 71 hours. For example, the dose for administration during the first 72 hours of the continuous infusion can be administered at two rates such that at least 13% of the dose is administered in the first hour, and the remainder of the dose (87% of the dose) for the first 72 hours is administered in the remaining 71 hours. For example, the dose for administration during the first 72 hours of the continuous infusion can be administered at two rates such that at least 14% of the dose is administered in the first hour, and the remainder of the dose (86% of the dose) for the first 72 hours is administered in the remaining 71 hours. For example, the dose for administration during the first 72 hours of the continuous infusion can be administered at two rates such that at least 15% of the dose is administered in the first hour, and the remainder of the dose (85% of the dose) for the first 72 hours is administered in the remaining 71 hours. In certain embodiments, at least 16%, 17%, 18%, 19%, 20%, 21%, 22%, 23%, 24% or 25% of the dose is administered in the first hour, with the remainder being administered in the subsequent 71 hours.

In certain embodiments about 5%, about 6%, about 7%, about 8%, about 8.1%, about 8.2%, about 8.3%, about 8.4%, about 8.5%, about 8.6%, about 8.7%, about 8.8%, about 8.9% about 9%, about 10%, about 11%, about 12%, about 13%, about 14%, about 15%, about 16%, about 17%, about 18%, about 19%, about 20% about 21% about 22%, about 23%, about 24%, or about 25% of the dose is administered in the first hour, and the remainder of the dose for the first 72 hours, for example, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, or about 75% is administered in the subsequent 71 hours. Any of these values may be used to define a range for the percentage of the dose that is administered in the first hour and the percentage of the dose administered in the subsequent 71 hours. For example, the percentage of the dose that is administered in the first hour may range from 5% to 25%, 5% to 20%, 5% to 15%, 5% to 10%, 10% to 25%, 10% to 20%, 10% to 15%, 15% to 25%, 15% to 20%, 20% to 25%, 8% to 25%, 8% to 15%, 6% to 9%, or 7% to 9%. The remaining dose is administered in the subsequent 71 hours. For example, the percentage of the dose that is administered in the subsequent 71 hours may range from 75% to 95%, 75% to 90%, 75% to 85%, 75% to 80%, 80% to 95%, 80% to 90%, 80% to 85%, 85% to 95%, 85% to 90%, 90% to 95%, 91% to 94%, 91% to 93%, from 75% to 92%, or from 85% to 92%.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, a “formulation” is understood as an active ingredient, e.g., CoQ10, in combination with any pharmaceutically acceptable carrier. Formulations can include, but are not limited to, aqueous formulations, liposomal formulations, suspensions, emulsions, microemulsions, nanoemulsions, nanosuspensions, formulations for specific routes of administration for injection or infusion.

As used herein, the term “safe and therapeutic effective amount” refers to the quantity of a component which is sufficient to yield a desired therapeutic response without undue adverse side effects (such as toxicity, irritation, or allergic response) commensurate with a reasonable benefit/risk ratio when used in the manner of this disclosure. By “therapeutically effective amount” is meant an amount of a compound of the present disclosure effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives. The “therapeutically effective amount” will vary depending on the compound, the disease and its severity and the age, weight, etc., of the patient to be treated.

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like.

“Adverse events” or “AEs” are characterized by grade depending on the severity. Some AE (e.g., nausea, low blood counts, pain, reduced blood clotting) can be treated so that the specific chemotherapeutic regimen can be continued or resumed. Some adverse events (e.g., loss of cardiac, liver, or kidney function; nausea) may not be treatable, requiring termination of treatment with the drug. Determination of AE grade and appropriate interventions can be determined by those of skill in the art. Common Terminology Criteria for Adverse Events v4.0 (CTCAE) (Publish Date: May 28, 2009) provide a grading scale for adverse events as follows:

Grade 1—Mild; asymptomatic or mild symptoms; clinical or diagnostic observations only; intervention not indicated.

Grade 2—Moderate; minimal, local or noninvasive intervention indicated; limiting age-appropriate instrumental activities of daily life (ADL).

Grade 3—Severe or medically significant but not immediately life-threatening;

hospitalization or prolongation of hospitalization indicated; disabling, limiting self care ADL.

Grade 4—Life-threatening consequences; urgent intervention indicated.

Grade 5—Death related to adverse event.

Adverse events include “coagulopathy” or “reduced blood clotting.” Methods to determine clotting times are well known and typically are based on determination of the “international normalized ratio” or “INR” which is a ratio based on the prothrombin time (in seconds) for a normal individual. The prothrombin time is the time it takes plasma to clot after addition of tissue factor. The INR was devised to standardize clotting time results between individuals and tissue factor used in the assay prepared by different manufacturers. Each manufacturer assigns an ISI value (International Sensitivity Index) for any tissue factor each batch of tissue factor manufactured. The ISI value indicates how a particular batch of tissue factor compares to an international reference tissue factor. The ISI is usually between 1.0 and 2.0. The INR is the ratio of a patient's prothrombin time to a normal (control) sample, raised to the power of the ISI value for the analytical system used. Such methods are routine in the art. The INR is typically used to monitor patients on anticoagulant therapy, e.g., warfarin therapy, or being treated with other agents, e.g., agents for the treatment of cancer including coenzyme Q10. The normal range for a healthy person not using warfarin is 0.8-1.2, and for people on warfarin therapy an INR of 2.0-3.0 usually targeted. Therefore, an elevation of INR over that observed in a healthy person (e.g., up to 1.5, 2.0. 2.5, or 3.0) is not necessarily considered to be an adverse event requiring intervention. Such considerations are understood by those of skill in the art. If the INR is outside the target range, a high INR indicates a higher risk of bleeding, while a low INR suggests a higher risk of developing a clot. Other methods to assess clotting times are known in the art.

As used herein, “co-administration” or “combination therapy” is understood as administration of two or more active agents using separate formulations or a single pharmaceutical formulation, or consecutive administration in any order such that, there is a time period while both (or all) active agents simultaneously exert their biological activities. Co-administration does not require that the agents are administered at the same time, at the same frequency, or by the same route of administration. As used herein, “co-administration” or “combination therapy” includes administration of a CoQ10 compound with one or more additional anti-cancer agents, e.g., chemotherapeutic agents, or administration of two or more CoQ10 compounds. Examples of anticancer agents, including chemotherapeutic agents, are provided herein.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein including, but not limited to, combinations of dosing rates, dosing times, dosing amounts, treatment methods, monitoring methods, selection methods, and use of agents other than coenzyme Q10.

### II. Coenzyme 010

Coenzyme Q10 has the following structure:

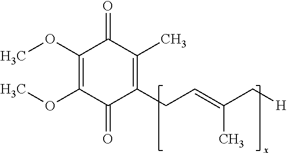

wherein x is 10. Coenzyme Q10 includes the fully oxidized version, also known as ubiquinone, the partially oxidized version, also known as semiquinone or ubisemiquinone, or the fully reduced version, also known as ubiquinol; or any mixtures or combinations thereof. In certain embodiments, the CoQ10 for treatment of pancreatic cancer is ubiquinone. In certain embodiments, the CoQ10 for treatment of pancreatic cancer is ubiquinol.

Coenzyme Q10, also referred to herein as CoQ10, is also known as ubidecarenone. Coenzyme Q10 is art-recognized and further described in International Publication No. WO 2005/069916 (Appln. No. PCT/US2005/001581, WO 2008/116135 (Appln. No. PCT/US08/57786), WO2010/132507 (Appln. No. PCT/US2010/034453), WO 2011/112900 (Appln. No. PCT/US2011/028042), and WO2012/174559 (Appln. No. PCT/US2012/043001) the entire contents of each of which are expressly incorporated by reference herein. Coenzyme Q10 is one of a series of polyprenyl 2,3-dimethoxy-5-methylbenzoquinone (ubiquinone) present in the mitochondrial electron transport systems of eukaryotic cells. Human cells produce coenzyme Q10 exclusively and it is found in cell and mitochondrial membranes of all human cells, with the highest levels in organs with high energy requirements, such as the liver and the heart. The body pool of coenzyme Q10 has been estimated to be about 2 grams, of which more than 50% is endogenous. Approximately 0.5 grams of coenzyme Q10 is required from the diet or biosynthesis each day. Coenzyme Q10 is produced in ton quantities from the worldwide supplement market and can be obtained from Kaneka, with plants in Pasadena, Tex. and Takasagoshi, Japan.

### III. Compositions

The present disclosure provides compositions containing CoQ10 for the treatment and prevention of pancreatic cancer. The compositions of the present disclosure can be self-administered by a patient, or in pharmaceutical compositions where it is mixed with suitable carriers or excipient(s). In treating a patient exhibiting a disorder of interest, e.g., pancreatic cancer, a therapeutically effective amount of the CoQ10 is administered. A therapeutically effective dose refers to that amount of the compound which results in at least stable disease or a prolongation of survival in a patient.

Suitable routes of administration of the present compositions of the invention may include parenteral delivery, including, intravenous infusion, preferably continuous infusion. In a preferred embodiment, the IV infusion comprises the active agent, e.g., coenzyme Q10, at approximately a 40 mg/mL (4% w/v) concentration. Where the composition is administered by IV infusion, it can be diluted in a pharmaceutically acceptable aqueous solution such as phosphate buffered saline or normal saline. In some embodiments, one or more routes of administration may be combined, such as, for example, intravenous and intratumoral, or intravenous and peroral, or intravenous and oral, or intravenous and topical, transdermal, or transmucosal. However, the methods provided herein include administration of coenzyme Q10 by continuous intravenous infusion.

For example, CoQ10 can be formulated for parenteral delivery, e.g., for intravenous injection. The compositions may be administered in a single bolus, multiple injections, or by continuous infusion (for example, intravenously or by peritoneal dialysis). For parenteral administration, the compositions may be formulated in a sterilized pyrogen-free form.

Use of pharmaceutically acceptable carriers to formulate the compounds herein disclosed, for the practice of the present invention, into dosages suitable for systemic administration is within the scope of the present disclosure. With proper choice of carrier and suitable manufacturing practice, the compositions of the present disclosure, in particular, those formulated as solutions, may be administered parenterally, such as by intravenous injection.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices may be desirable. The data obtained from these cell culture assays and animal studies can be used in formulating a range of dosage for use in human. The dosage of such compounds may be within a range of circulating concentrations that include the ED50 with little or no toxicity. The dosage may vary within this range depending upon the dosage form employed and the route of administration utilized.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for intravenous administration may be in the form of solutions of colloidal dispersion.

Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### IV. Formulations

CoQ10 can be delivered in any pharmaceutically acceptable carrier for the desired route of administration. As used herein, formulations including CoQ10 are formulated for administration by injection or infusion.

Preferred therapeutic formulations for use in the methods of the invention comprise CoQ10 in a microparticle formation, e.g., for intravenous administration. Such intravenous formulations are provided, for example, in WO2011/112900 (Appln. No. PCT/US2011/028042), the entire contents of which are expressly incorporated herein by reference, and an exemplary intravenous formulation as described in WO2011/112900 (Appln. No. PCT/US2011/028042) is used in the examples set forth below. Through high pressure homogenization, CoQ10 particles are reduced to produce particles that are small enough to pass through a 200-nm sterilizing filter. Particles that are small enough to pass through a 200-nm sterilizing filter can be injected intravenously. These particles are much smaller than blood cells and therefore will not embolize capillaries. Red blood cells for example are 6-micron×2-micron disks. The particles are dispersed to and are encased or surrounded by a stabilizing agent. While not wishing to be bound by any theory, it is believed that the stabilizing agents are attracted to the hydrophobic therapeutic agent such that the dispersed particles of the hydrophobic therapeutic agent are surrounded by the stabilizing agent forming a suspension or an emulsion. The dispersed particles in the suspension or emulsion comprises a stabilizing agent surface and a core consisting of the hydrophobic therapeutic agent, e.g., CoQ10, in a solid particulate form (suspension) or in an immiscible liquid form (emulsion). The dispersed particles can be entrenched in the lipophilic regions of a liposome.

Dispersed colloidal systems permit a high drug load in the formulation without the use of co-solvents. Additionally, high and relatively reproducible plasma levels are achieved without the dependence on endogenous low-density lipoprotein carriers. More importantly, the formulations allow sustained high drug levels in tumor cells due to the passive accumulation of the colloidal particles of the hydrophobic therapeutic agent.

A preferred intravenous formulation substantially comprises a continuous phase of water and dispersed solids (suspension) or dispersed immiscible liquid (emulsion). Dispersed colloidal systems, in which the particles are composed largely of the active agent (drug) itself, can often deliver more drug per unit volume than continuous solubilizing systems, if the system can be made adequately stable.

As the formulation medium, the aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. For example, aqueous solutions can be used to dilute a formulation for intravenous administration from a concentration of about 4% w/v to a lower concentration to facilitate administration of lower doses of coenzyme Q10. The aqueous solution may contain substances which increase the viscosity of the solution, such as sodium carboxymethyl cellulose, sorbitol, or dextran.

CoQ10 is dispersed in the aqueous solution such that a colloidal dispersion is formed wherein the nano-dispersion particles of the hydrophobic therapeutic agent are covered or encased or encircled by the dispersion stabilizing agents to form nano-dispersions of the CoQ10 compound particles. The nano-dispersed CoQ10 particles have a core formed of the hydrophobic therapeutic agent that is surrounded by the stabilizing agent. Similarly, in certain aspects, the stabilizing agent is a phospholipid having both a hydrophilic and lipophilic portion. The phospholipids form liposomes or other nanoparticles upon homogenization. In certain aspects these liposomes are bi-layered unilamellar liposomes while in other embodiments the liposomes are bi-layered multi-lamellar liposomes. The dispersed CoQ10 particles are dispersed in the lipophilic portion of the bi-layered structure of the liposome formed from the phospholipids. In certain other aspects the core of the liposome, like the core of the nano-dispersion of CoQ10 particles, is formed of the hydrophobic therapeutic agent and the outer layer is formed of the bi-layered structure of the phospholipid. In certain embodiments the colloidal dispersions are treated by a lyophilization process whereby the nanoparticle dispersion is converted to a dry powder.

In some embodiments, the formulation for injection or infusion used is a 4% sterile aqueous colloidal dispersion containing coenzyme Q10 in a nanosuspension as prepared in WO2011/112900. In certain embodiments, the formulation includes an aqueous solution, coenzyme Q10, and at least one of a dispersion stabilizing agent and an opsonization reducer; wherein the colloidal nano-dispersion of the active agent is dispersed into nano-dispersion particles having a mean size of less than 200 nm.

In certain embodiments, the dispersion stabilizing agent includes, but is not limited to, pegylated castor oil, Cremphor® EL, Cremophor® RH 40, Pegylated vitamin E, Vitamin E TPGS, and Dimyristoylphosphatidyl choline (DMPC).

In certain embodiments, the opsonization reducer is a poloxamer or a poloxamines.

In certain embodiments, the colloidal nano-dispersion is a suspension or an emulsion. Optionally, a colloidal nano-dispersion is in a crystalline form or a super-cooled melt form.

In certain embodiments, the formulation for injection or infusion includes a lyoprotectant such as a nutritive sugar including, but not limited to, lactose, mannose, maltose, galactose, fructose, sorbose, raffinose, neuraminic acid, glucosamine, galactosamine, N-methylglucosamine, mannitol, sorbitol, arginine, glycine, and sucrose; or any combination thereof.

In certain embodiments, the formulation for injection or infusion includes an aqueous solution; a hydrophobic active agent dispersed to form a colloidal nano-dispersion of particles; and at least one of a dispersion stabilizing agent and an opsonization reducer. The colloidal nano-dispersion of the active agent is dispersed into nano-dispersion particles having sizes of less than 200 nm. In some embodiments the dispersion stabilizing agent is selected from natural or semisynthetic phospholipids. For example, suitable stabilizing agents include polyethoxylated (a/k/a pegylated) castor oil (Cremophor® EL), polyethoxylated hydrogenated castor oil (Cremophor® RH 40), Tocopherol polyethylene glycol succinate (Pegylated vitamin E, Vitamin E TPGS), Sorbitan fatty acid esters (Spans®), bile acids and bile-acid salts, or dimyristoylphosphatidyl choline (DMPC). In some embodiments the stabilizing agent is DMPC.

In certain embodiments the formulation is suitable for parenteral administration, including intravenous, intraperitoneal, orthotopical, intracranial, intramuscular, subcutaneous, intramedullary injections, as well as intrathecal, direct intraventricular, intranasal, or intraocular injections. In certain embodiments, the formulation contains coenzyme Q10, dimyristoyl-phophatidylcholine, and poloxamer 188 in a ratio of 4:3:1.5 respectively that is designed to stabilize the nanosuspension of the particles. In some embodiments, the formulation includes a phosphate buffer saline solution which contains sodium phosphate dibasic, potassium phosphate monobasic, potassium chloride, sodium chloride, and water for injection. In certain embodiments, the 4% sterile aqueous colloidal dispersion containing coenzyme Q10 in a nanosuspension is diluted in the phosphate buffered saline solution provided, e.g., 1:1, 1:2, 1:3, 1:4. 1:5, 1:6, 1:7, 1:8. 1:9, 1:10, 1:11, 1:12, 1:13, 1:14. 1:15, 1:16, 1:17, 1:18. 1:19, 1:20, or other appropriate ratio bracketed by any two of the values.

In some embodiments, a formulation for administration for use in the invention may include from about 0.001% to about 20% (w/w) of coenzyme Q10, about 0.01% to about 20% (w/w) of coenzyme Q10, about 0.1% to about 20% (w/w) of coenzyme Q10, more preferably about 0.01% to about 15% and even more preferably about 0.1% to about 10% (w/w) of coenzyme Q10. In certain embodiments, a formulation for any route of administration for use in the invention may include from about 1% to about 10% (w/w) of coenzyme Q10. In certain embodiments, a formulation for any route of administration for use in the invention may include from about 2% to about 8% (w/w) of coenzyme Q10. In certain embodiments, a formulation for any route of administration for use in the invention may include from about 2% to about 7% (w/w) of coenzyme Q10. In certain embodiments, a formulation for any route of administration for use in the invention may include from about 3% to about 6% (w/w) of coenzyme Q10. In certain embodiments, a formulation for any route of administration for use in the invention may include from about 3% to about 5% (w/w) of coenzyme Q10. In certain embodiments, a formulation for any route of administration for use in the invention may include from about 3.5% to about 4.5% (w/w) of coenzyme Q10. In certain embodiments, a formulation for any route of administration for use in the invention may include from about 3.5% to about 5% (w/w) of coenzyme Q10. In one embodiment a formulation includes about 4% (w/w) of coenzyme Q10. In one embodiment a formulation includes about 8% (w/w) of coenzyme Q10. In various embodiments, the formulation includes about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 7%, 8%, 9%, 10%, 11%, 12%, 13%, 14%, 15%, 16%, 17%, 18%, 19% or 20% (w/w) of coenzyme Q10, or any range bracketed by any two values recited. In certain embodiments, the formulations can be prepared as a percent weight to volume rather than a percent weight to weight. Depending on the formulation, the concentration of coenzyme Q10 may be the same, or about the same in the w/w and the w/v percent formulations. Coenzyme Q10 can be obtained from Kaneka Q10 as Kaneka Q10 (USP UBIDECARENONE) in powdered form (Pasadena, Tex., USA). Coenzyme Q10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2% less than; heavy metal content is less than 0.002%, or less than 0.001%; purity of between 98-100% or 99.9%, or 99.5%.

In certain embodiments, the concentration of coenzyme Q10 in the formulation is 1 mg/mL to 150 mg/mL. In one embodiment, the concentration of coenzyme Q10 in the formulation is 5 mg/mL to 125 mg/mL. In one embodiment, the concentration of coenzyme Q10 in the formulation is 10 mg/mL to 100 mg/mL. In one embodiment, the concentration of coenzyme Q10 in the formulation is 20 mg/mL to 90 mg/mL. In one embodiment, the concentration of coenzyme Q10 is 30 mg/mL to 80 mg/mL. In one embodiment, the concentration of coenzyme Q10 is 30 mg/mL to 70 mg/mL. In one embodiment, the concentration of coenzyme Q10 is 30 mg/mL to 60 mg/mL. In one embodiment, the concentration of coenzyme Q10 is 30 mg/mL to 50 mg/mL. In one embodiment, the concentration of coenzyme Q10 is 35 mg/mL to 45 mg/mL. It should be understood that additional ranges having any one of the foregoing values as the upper or lower limits are also intended to be part of this invention, e.g., 10 mg/mL to 50 mg/mL, or 20 mg/mL to 60 mg/mL.

In certain embodiments, the concentration of coenzyme Q10 in the formulation is about 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 mg/mL. In one embodiment, the concentration of coenzyme Q10 in the formulation is about 50 mg/mL. In one embodiment, the concentration of coenzyme Q10 in the formulation is about 60 mg/mL. In one embodiment, the concentration of coenzyme Q10 in the formulation is about 30 mg/mL. In a preferred embodiment, the concentration of coenzyme Q10 in the formulation is about 40 mg/mL. It should be understood that ranges having any one of these values as the upper or lower limits are also intended to be part of this invention, e.g. between 37 mg/mL and 47 mg/mL, or between 31 mg/mL and 49 mg/mL.

It is understood that formulations can similarly be prepared containing coenzyme Q10 precursors, metabolites, and related compounds.

### V. Treatment of Pancreatic Cancer

The invention provides methods for the treatment of pancreatic cancer in a population of subjects in need thereof, the method comprising administering to each subject of the population a composition comprising Coenzyme Q10. In some embodiments, the pancreatic cancer is metastatic. In some embodiments, the composition comprising Coenzyme Q10 is administered by continuous infusion, e.g. continuous intravenous infusion. In certain embodiments, the pancreatic cancer is treated with coenzyme Q10 alone. In certain embodiments, the pancreatic cancer is treated with coenzyme Q10 and an additional agent. In certain embodiments, the additional agent is a chemotherapeutic agent. In certain embodiments, treatment with the chemotherapeutic agent is initiated at the same time as treatment with the coenzyme Q10. In certain embodiments, the treatment with the chemotherapeutic agent is initiated after the treatment with coenzyme Q10 is initiated. In certain embodiments, treatment with the additional agent is initiated upon progression of the cancer during treatment with coenzyme Q10. In certain embodiments, treatment with the additional agent is initiated without progression of the cancer during treatment with coenzyme Q10. In certain embodiments, treatment with coenzyme Q10 is continued upon initiation of administration of the additional agent. In certain embodiments, treatment with coenzyme Q10 is stopped upon initiation of treatment with the additional agent.

In certain embodiments, at least 10%, 20%, 30%, 40%, 50%, 53%, 60%, 70%, 80%, 90% or 95% of the evaluable subjects in the population exhibit stable disease as a result of administration of the Coenzyme Q10 to the subjects, thereby treating the pancreatic cancer in the population of subjects. In some embodiments, at least 5%, 10%, 15%, 20%, 23%, 25%, 30%, 35%, 40%, 50%, 60%, 70%, 80%, 90% or 95% of the evaluable subjects in the population exhibit stable disease for at least four months as a result of administration of the Coenzyme Q10 to the subjects having pancreatic cancer. In some embodiments, the overall population of subjects exhibits a median progression free survival of at least 50, 60, 70, 80, 90, 100, 110, 120, 130, 140, 150, 160, 170, 180, 188, 190, 200, 210, 220, 230, 240 or 250 days as a result of administration of the Coenzyme Q10 to the subjects having pancreatic cancer. In some embodiments, the population of subjects exhibits a median overall survival of at least 50, 60, 70, 80, 90, 100, 110, 120, 125, 150, 175, 200, 218, 225, 250, 275 or 300 days as a result of administration of the Coenzyme Q10 to the subjects having pancreatic cancer. In some embodiments, the population of subjects exhibits a median time to progression of at least 10, 20, 30, 40, 50, 60, 65, 68, 70, 80, 90, 100, 110, 120, 121, 130, 140, 150, 160, 170, 180, 190 or 200 days as a result of administration of the Coenzyme Q10 to the subjects having pancreatic cancer.

In certain embodiment, formulations and methods of the present disclosure may be utilized for the treatment of pancreatic cancer wherein the subject has failed at least one prior chemotherapeutic regimen. Accordingly, the present invention provides methods of treating pancreatic cancer in a subject, wherein the subject has failed at least one prior chemotherapeutic regimen for the cancer, comprising administering the formulations of the invention to the subject by continuous infusion in an amount sufficient to treat the pancreatic cancer, thereby treating the pancreatic cancer. In certain embodiments, the subject is a human subject.

In certain embodiments of the invention, methods are provided for treating or preventing pancreatic cancer in a human by intravenously administering coenzyme Q10 to the human such that treatment or prevention occurs, wherein the human is administered the coenzyme Q10 compound, e.g., coenzyme Q10, by continuous infusion (e.g., for at least 24 hours, at least 48 hours, at least 72 hours, at least 96 hours or at least 144 hours) wherein a 24 hour dose (e.g., average 24 hour dose) of the coenzyme Q10 compound (e.g., coenzyme Q10) is administered in the range of about 0.5 mg/kg/dose to about 10,000 mg/kg/dose, about 5 mg/kg/dose to about 5,000 mg/kg/dose, about 10 mg/kg/dose to about 3,000 mg/kg/dose. In one embodiment, the formulation is administered such that, preferably, coenzyme Q10 is administered a 24 hour dose in the range of about 10 mg/kg/dose to about 1,400 mg/kg/dose. In one embodiment, the formulation is administered a 24 hour dose such that, preferably, coenzyme Q10 is administered in the range of about 10 mg/kg/dose to about 650 mg/kg/dose. In one embodiment, the formulation is administered such that, preferably, coenzyme Q10 is administered in the range of about 10 mg/kg/dose to about 200 mg/kg/dose. In one embodiment, the formulation is administered a 24 hour dose such that, preferably, coenzyme Q10 is administered in the range of about 10 mg/kg/dose to about 100 mg/kg/dose. In one embodiment, the formulation is administered a 24 hour dose such that, preferably, coenzyme Q10 is administered in the range of about 10 mg/kg/dose to about 75 mg/kg/dose. In one embodiment, the formulation is administered a 24 hour dose such that, preferably, coenzyme Q10 is administered in the range of about 10 mg/kg/dose to about 65 mg/kg/dose. In various embodiments, the formulation is administered a 24 hour dose such that, preferably, coenzyme Q10 is administered in the range of about 10 mg/kg/dose to about 15 mg/kg/dose, about 15 mg/kg/dose to about 20 mg/kg/dose, about 20 mg/kg/dose to about 25 mg/kg/dose, about 27 mg/kg/dose to about 35 mg/kg/dose, about 34 mg/kg/dose to about 42 mg/kg/dose, about 42 mg/kg/dose to about 52 mg/kg/dose, or about 53 mg/kg/dose to about 65 mg/kg/dose. In various embodiments, the formulation is administered such that, preferably, coenzyme Q10 is administered a 24 hour dose at a dose of about 2 mg/kg/dose, 5 mg/kg/dose, 10 mg/kg/dose, 15 mg/kg/dose, 20 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 65 mg/kg/dose, 70 mg/kg/dose, 75 mg/kg/dose, 76 mg/kg/dose, 77 mg/kg/dose, 78 mg/kg/dose, 79 mg/kg/dose, 80 mg/kg/dose, 85 mg/kg/dose, 90 mg/kg/dose, 95 mg/kg/dose, 100 mg/kg/dose, 101 mg/kg/dose, 102 mg/kg/dose, 103 mg/kg/dose, 104 mg/kg/dose, 105 mg/kg/dose, 106 mg/kg/dose, 107 mg/kg/dose, 108 mg/kg/dose, 109 mg/kg/dose, 110 mg/kg/dose, 120 mg/kg/dose, 130 mg/kg/dose, 140 mg/kg/dose, 150 mg/kg/dose, 160 mg/kg/dose, 170 mg/kg/dose, 180 mg/kg/dose, 190 mg/kg/dose, or 200 mg/kg/dose. In various embodiments, the formulation is administered a 24 hour dose such that, preferably, coenzyme Q10 is administered at a dose of at least 2 mg/kg/dose, 5 mg/kg/dose, 10 mg/kg/dose, 15 mg/kg/dose, 20 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 65 mg/kg/dose, 70 mg/kg/dose, 75 mg/kg/dose, 76 mg/kg/dose, 77 mg/kg/dose, 78 mg/kg/dose, 79 mg/kg/dose, 80 mg/kg/dose, 85 mg/kg/dose, 90 mg/kg/dose, 95 mg/kg/dose, 100 mg/kg/dose, 101 mg/kg/dose, 102 mg/kg/dose, 103 mg/kg/dose, 104 mg/kg/dose, 105 mg/kg/dose, 106 mg/kg/dose, 107 mg/kg/dose, 108 mg/kg/dose, 109 mg/kg/dose, 110 mg/kg/dose, 120 mg/kg/dose, 130 mg/kg/dose, 140 mg/kg/dose, 150 mg/kg/dose, 160 mg/kg/dose, 170 mg/kg/dose, 180 mg/kg/dose, 190 mg/kg/dose, or 200 mg/kg/dose, wherein the dose does not result in any limiting toxicities. It should be understood that ranges having any one of these values as the upper or lower limits of a 24 hour dose are also intended to be part of this invention, e.g., about 50 mg/kg/dose to about 200 mg/kg/dose, or about 650 mg/kg/dose to about 1400 mg/kg/dose, or about 55 mg/kg/dose to about 110 mg/kg/dose. In one embodiment the administered 24 hour dose is at least about 1 mg/kg/dose, at least about 5 mg/kg/dose, at least about 10 mg/kg/dose, at least about 12.5 mg/kg/dose, at least about 15 mg/kg/dose, at least about 20 mg/kg/dose, at least about 25 mg/kg/dose, at least about 30 mg/kg/dose, at least about 35 mg/kg/dose, at least about 40 mg/kg/dose, at least about 45 mg/kg/dose, at least about 50 mg/kg/dose, at least about 55 mg/kg/dose, at least about 60 mg/kg/dose, at least about 65 mg/kg/dose, at least about 75 mg/kg/dose, at least about 100 mg/kg/dose, at least about 125 mg/kg/dose, at least about 150 mg/kg/dose, at least about 175 mg/kg/dose, at least about 200 mg/kg/dose, at least about 300 mg/kg/dose, or at least about 400 mg/kg/dose. In certain embodiments, the administered 24 hour dose is no more than about 20 mg/kg/dose, about 25 mg/kg/dose, about 30 mg/kg/dose, about 35 mg/kg/dose, about 40 mg/kg/dose, about 45 mg/kg/dose, about 50 mg/kg/dose, about 55 mg/kg/dose, about 60 mg/kg/dose, about 75 mg/kg/dose, about 100 mg/kg/dose, about 125 mg/kg/dose, about 150 mg/kg/dose, about 175 mg/kg/dose, about 200 mg/kg/dose, about 300 mg/kg/dose, about 400 mg/kg/dose, about 500 mg/kg/dose, about 600 mg/kg/dose, about 700 mg/kg/dose, about 800 mg/kg/dose, about 900 mg/kg/dose, about 1000 mg/kg/dose, about 1100 mg/kg/dose, about 1200 mg/kg/dose, or about 1300 mg/kg/dose. It is understood that any of the lower limit values and upper limit values can be combined to create a range.

In certain embodiments, a dose can be a 48 hour dose (i.e., about 2 days) administered by continuous infusion in any of the foregoing amounts or ranges of amounts provided. In certain embodiments, the total 48 hour dose administered by continuous infusion is 2 times the amount administered in a 24 hour dose. In certain embodiments, the total 48 hour dose administered by continuous infusion is administered over 48 hours with an average dose over 24 hours of any of the foregoing amounts or ranges of amounts provided. In certain embodiments, the total 48 hour dose administered by continuous infusion is equivalent to any of the foregoing 24 hour doses, but is administered over 48 hours. In certain embodiments the total 48 hour dose is about 30 mg/kg to about 350 mg/kg, or about 30 mg/kg to about 300 mg/kg. In certain embodiments, the total 48 hour continuous infusion dose is about 50 mg/kg to about 250 mg/kg. In certain embodiments, the total 48 hour continuous infusion dose is about 38 mg/kg, about 50 mg/kg, about 66 mg/kg, about 88 mg/kg, about 110 mg/kg, about 137 mg/kg, about 171 mg/kg, or about 215 mg/kg.

In certain embodiments, a dose can be a 96 hour dose (i.e., about 4 days) administered by continuous infusion in any of the amounts or ranges of amounts provided. In certain embodiments, the total 96 hour dose administered by continuous infusion is four times the amount administered in a 24 hour dose. In certain embodiments, the 96 hour dose administered by continuous infusion is administered over 96 hours with an average dose over 24 hours of any of the foregoing amounts or ranges of amounts provided. In certain embodiments, the total 96 hour dose administered by continuous infusion is equivalent to any of the foregoing 24 hour doses, but is administered over 96 hours. In certain embodiments the total 96 hour dose is about 30 mg/kg to about 450 mg/kg, 30 mg/kg to about 400 mg/kg, about 30 mg/kg to about 350 mg/kg, or about 30 mg/kg to about 300 mg/kg. In certain embodiments, the total 96 hour continuous infusion dose is about 50 mg/kg to about 250 mg/kg. In certain embodiments the total 96 hour dose is about 100 mg/kg to about 430 mg/kg, 100 mg/kg to about 200 mg/kg, about 200 mg/kg to about 350 mg/kg, or about 300 mg/kg to about 450 mg/kg. In certain embodiments, the total 96 hour continuous infusion dose is about 38 mg/kg, about 50 mg/kg, about 66 mg/kg, about 88 mg/kg, about 110 mg/kg, about 137 mg/kg, about 171 mg/kg, about 215 mg/kg, about 100 mg/kg, about 132 mg/kg, about 176 mg/kg, about 220 mg/kg, about 274 mg/kg, about 342 mg/kg, or about 430 mg/kg.

In certain embodiments, a dose can be a 72 hour dose (i.e., about 3 days) administered by continuous infusion in any of the amounts or ranges of amounts provided. In certain embodiments, the total 72 hour dose administered by continuous infusion is 3 times the amount administered in a 24 hour dose. In certain embodiments, the 72 hour dose administered by continuous infusion is administered over 72 hours with an average dose over 24 hours of any of the foregoing amounts or ranges of amounts provided. In certain embodiments, the total 72 hour dose administered by continuous infusion is equivalent to any of the foregoing 24 hour doses, but is administered over 72 hours. In certain embodiments the total 72 hour dose is about 30 mg/kg to about 350 mg/kg, or about 30 mg/kg to about 300 mg/kg. In certain embodiments, the total 72 hour continuous infusion dose is about 50 mg/kg to about 250 mg/kg. In certain embodiments, the total 72 hour continuous infusion dose is about 38 mg/kg, about 50 mg/kg, about 66 mg/kg, about 88 mg/kg, about 110 mg/kg, about 137 mg/kg, about 171 mg/kg, or about 215 mg/kg.

In certain embodiments, a dose can be a 144 hour dose (i.e., about 6 days) administered by continuous infusion in any of the foregoing amounts or ranges of amounts provided. In certain embodiments, the 144 hour dose of Coenzyme Q10 is administered to the subject once per week for at least 1 week. In certain embodiments, the 144 hour dose of Coenzyme Q10 is administered to the subject once per week for at least 2 weeks. In certain embodiments, the 144 hour dose of Coenzyme Q10 is administered to the subject once per week for at least 3 weeks. In certain embodiments, the 144 hour dose of Coenzyme Q10 is administered to the subject once per week for at least 4 weeks. In certain embodiments, the 144 hour dose of Coenzyme Q10 is administered to the subject once per week for at least 5 weeks. In certain embodiments, the 144 hour dose of Coenzyme Q10 is administered to the subject once per week for at least 6 weeks. In certain embodiments, the 144 hour dose of Coenzyme Q10 is administered to the subject once per week for at least 7 weeks. In certain embodiments, the 144 hour dose of Coenzyme Q10 is administered to the subject once per week for at least 8 weeks. In certain embodiments, the 144 hour dose of Coenzyme Q10 is administered to the subject once per week for at least 9 weeks. In certain embodiments, the 144 hour dose of Coenzyme Q10 is administered to the subject once per week for at least 10 weeks.

In certain embodiments, the subject (e.g., evaluable subject) receives Coenzyme Q10 treatment for at least 30 days and at least one dose of gemcitabine. In certain embodiments, the subject (e.g., evaluable subject) receives Coenzyme Q10 treatment for at least 30 days over a 10 week period, and at least one dose of gemcitabine. In some embodiments, the 30 days are not consecutive. For example, in some embodiments, the subject receives at least one dose of gemcitabine and one 144 hour (6 day) continuous intravenous infusion of Coenzyme Q10 for at least 5 weeks (e.g., 5 consecutive weeks or 5 on-consecutive weeks). For example, in some embodiments, the subject receives at least one dose of gemcitabine and one 144 hour (6 day) continuous intravenous infusion of Coenzyme Q10 per week for 5, 6, 7, 8, 9 or 10 weeks over a 10 week period. In certain embodiments, the subject receives at least one dose of gemcitabine and Coenzyme Q10 treatment for at least 30 days over a period of two cycles, wherein the two cycles consist of a first cycle comprising administering a 144 hour continuous intravenous infusion of Coenzyme Q10 once per week for 6 weeks, and a second cycle comprising administering a 144 hour continuous intravenous infusion of Coenzyme Q10 once per week for 4 weeks. The subject may not receive Coenzyme Q10 during every week of the cycle. For example, in some embodiments the subject receiving Coenzyme Q10 treatment for at least 30 days over a period of two cycles receives Coenzyme Q10 for at least 5 weeks (e.g., either consecutively or non-consecutively), at least 6 weeks (e.g., consecutively or non-consecutively), at least 7 weeks (e.g., either consecutively or non-consecutively), at least 8 weeks (e.g., either consecutively or non-consecutively), at least 9 weeks (e.g., either consecutively or non-consecutively) or at least 10 weeks (e.g., either consecutively or non-consecutively).

In certain embodiments, the subject (e.g., evaluable subject) receives Coenzyme Q10 treatment for at least 30 days and at least one dose of gemcitabine of at least 1000 mg/m². In some embodiments, the subject receives at least 2, 3, 4, 5 or 6 doses of gemcitabine, e.g. of at least 1000 mg/m². In certain embodiments, the subject (e.g., evaluable subject) receives Coenzyme Q10 treatment for at least 30 days over a 10 week period, and at least one dose of gemcitabine of at least 1000 mg/m². In some embodiments, the subject receives at least 2, 3, 4, 5 or 6 doses of gemcitabine, e.g. of at least 1000 mg/m². In some embodiments, the 30 days are not consecutive. For example, in some embodiments, the subject receives at least one dose of gemcitabine and one 144 hour (6 day) continuous intravenous infusion of Coenzyme Q10 for at least 5 weeks (e.g., 5 consecutive weeks or 5 on-consecutive weeks). In some embodiments, the subject receives at least 2, 3, 4, 5 or 6 doses of gemcitabine, e.g. of at least 1000 mg/m². For example, in some embodiments, the subject receives at least one dose of gemcitabine of at least 1000 mg/m² and one 144 hour (6 day) continuous intravenous infusion of Coenzyme Q10 per week for 5, 6, 7, 8, 9 or 10 weeks over a 10 week period. In certain embodiments, the subject receives at least one dose of gemcitabine of at least 1000 mg/m² and Coenzyme Q10 treatment for at least 30 days over a period of two cycles, wherein the two cycles consist of a first cycle comprising administering a 144 hour continuous intravenous infusion of Coenzyme Q10 once per week for 6 weeks, and a second cycle comprising administering a 144 hour continuous intravenous infusion of Coenzyme Q10 once per week for 4 weeks. In some embodiments, the subject receives at least 2, 3, 4, 5 or 6 doses of gemcitabine, e.g. of at least 1000 mg/m². The subject may not receive Coenzyme Q10 during every week of the cycle. For example, in some embodiments the subject receiving Coenzyme Q10 treatment for at least 30 days over a period of two cycles receives Coenzyme Q10 for at least 5 weeks (e.g., either consecutively or non-consecutively), at least 6 weeks (e.g., consecutively or non-consecutively), at least 7 weeks (e.g., either consecutively or non-consecutively), at least 8 weeks (e.g., either consecutively or non-consecutively), at least 9 weeks (e.g., either consecutively or non-consecutively) or at least 10 weeks (e.g., either consecutively or non-consecutively). The subject may not receive gemcitabine during every week of the cycle. For example, in some embodiments, the subject receives gemcitabine once weekly for at least 2 weeks (e.g., consecutively or non-consecutively), at least 3 weeks (e.g., consecutively or non-consecutively), at least 4 weeks (e.g., consecutively or non-consecutively), at least 5 weeks (e.g., consecutively or non-consecutively), or at least 6 weeks (e.g., consecutively or non-consecutively).

In certain embodiments, the total 144 hour dose administered by continuous infusion is 6 times the amount administered in a 24 hour dose. In certain embodiments, the 144 hour dose administered by continuous infusion is administered over 144 hours with an average dose over 24 hours of any of the foregoing amounts or ranges of amounts provided. In certain embodiments, the total 144 hour dose administered by continuous infusion is equivalent to any of the foregoing 24 hour doses, but is administered over 144 hours. In certain embodiments the total 144 hour dose is about 30 mg/kg to about 350 mg/kg, or about 30 mg/kg to about 300 mg/kg. In one embodiment the total 144 hour dose is about 75 mg/kg to about 350 mg/kg. In certain embodiments, the total 144 hour continuous infusion dose is about 50 mg/kg to about 250 mg/kg. In certain embodiments, the total 144 hour continuous infusion dose is about 38 mg/kg, about 50 mg/kg, about 66 mg/kg, about 88 mg/kg, about 110 mg/kg, about 137 mg/kg, about 171 mg/kg, or about 215 mg/kg. In certain embodiments, the total 144 hour continuous infusion dose is about 76 mg/kg, about 100 mg/kg, about 132 mg/kg, about 176 mg/kg, about 220 mg/kg, about 274 mg/kg, or about 342 mg/kg. In one embodiment, the coenzyme Q10 formulation is administered one time per week. In one embodiment, the coenzyme Q10 formulation is administered two times per week. In one embodiment, the coenzyme Q10 formulation is administered 3 times per week. In one embodiment, the coenzyme Q10 formulation is administered four times per week. In another embodiment, the coenzyme Q10 formulation is administered 5 times per week. In one embodiment, the coenzyme Q10 formulation is administered once per day. In certain embodiments, the formulation is administered over more than 4 hours. In certain embodiments, the formulation is administered over 8 or more hours. In certain embodiments, the formulation is administered over 12 hours or more hours. In certain embodiments, the formulation is administered over 18 or more hours. In certain embodiments, the formulation is administered over 24 or more hours (i.e., by continuous infusion). In certain embodiments, the formulation is administered over about 24 hours. In certain embodiments, the formulation is administered over at least about 48 hours, 72 hours, 96 hours, 120 hours, 144 hours, 168 hours, 192 hours, 216 hours, 240 hours, 264 hours, 288 hours, 312 hours, 336 hours, 360 hours, 384 hours, 408 hours, 432 hours, 456 hours, or 480 hours. In certain embodiments, the formulation is administered over about 48 hours, 72 hours, 96 hours, 120 hours, 144 hours, 168 hours, 192 hours, 216 hours, 240 hours, 264 hours, 288 hours, 312 hours, 336 hours, 360 hours, 384 hours, 408 hours, 432 hours, 456 hours, or 480 hours. Any of these values may be used to define a range for the number of hours over which the dose is administered. For example, the formulation may be administered over about 48 hours to about 480 hours, over about 48 hours to about 144 hours, or over about 48 hours to about 96 hours.

In certain embodiments, the amount of coenzyme Q10 administered is the same for each 24 hour period regardless of the rate(s) of administration during each 24 hour period of the continuous infusion. In certain embodiments, the amount of coenzyme Q10 administered varies for one or more 24 hour period of the continuous infusion. In certain embodiments, the amount of coenzyme Q10 administered during the first 24 hours is different from the amount of coenzyme Q10 administered during the remaining 24 hour periods in the continuous infusion. In certain embodiments, the amount of coenzyme Q10 administered is the same for all 24 hour periods, but the rate(s) of administration of coenzyme Q10 is different for the first 24 hour period as compared to the remaining 24 hour periods of the continuous infusion.

In certain embodiments, the continuous infusion is initiated with a loading dose of coenzyme Q10. In a preferred embodiment, the dosage administered in the 24, 48, or 72 hours is initially administered at a higher rate than the remainder of the 24, 48, or 72 hour infusion. In certain embodiments, at least 5% of the dose for the first 24, 48, or 72 hours is administered at the higher rate during the first hour of administration. In certain embodiments, at least 6% of the dose for the first 24, 48, or 72 hours is administered at the higher rate during the first hour of administration. In certain embodiments, at least 7% of the dose for the first 24, 48, or 72 hours is administered at the higher rate during the first hour of administration. In certain embodiments, at least 8% of the dose for the first 24, 48, or 72 hours is administered at the higher rate during the first hour of administration. In certain embodiments, at least 9% of the dose for the first 24, 48, or 72 hours is administered at the higher rate during the first hour of administration. In certain embodiments, at least 10% of the dose for the first 24, 48, or 72 hours is administered at the higher rate during the first hour of administration. In certain embodiments, at least 8% of the dose for the first 24, 48, or 72 hours is administered at the higher rate during the first hour of administration. In certain embodiments, at least 15% of the dose for the first 24, 48, or 72 hours is administered at the higher rate during the first hour of administration. In certain embodiments, at least 17.5% of the dose for the first 24, 48, or 72 hours is administered at the higher rate during the first hour of administration. In certain embodiments, at least 20% of the dose for the first 24, 48, or 72 hours is administered at the higher rate during the first hour of administration. In certain embodiments, at least 22.5% of the dose for the first 24, 48, or 72 hours is administered at the higher rate during the first hour of administration. In certain embodiments, at least 25% of the dose for the first 24, 48, or 72 hours is administered at the higher rate during the first hour of administration. After administration of a portion of the dose at a higher rate for the first of the 24, 48, or 72 hours, the remainder of the dose is administered at a second, lower rate for the remaining 23, −47, or 73 hours. Typically, the continuous infusion is continued to provide a continuous infusion of at least 96 hours (i.e. two 48 hour infusions with a short break between the two infusions) or at least 144 hours (i.e. two 72 hour infusions with a short break between the two infusions). This continuation may be at a third infusion rate, which is between the two prior infusion rates used such that the amount of coenzyme Q10 administered each 48 hours (for the 96 hour infusion) or each 72 hours (for the 144 hour infusion) is the same. Methods to adjust infusion rates to provide a loading dose at the beginning of the infusion are well within the ability of those of skill in the art.

In certain embodiments, the infusion rate for the loading dose for the first hour of the continuous administration is about 0.1 ml/min to about 1.5 ml/min. For example, the loading dose of the first hour of the infusion can be about 0.1 ml/min, 0.15 ml/min, 0.2 ml/min, 0.25 ml/min, 0.3 ml/min, 0.35 ml/min, 0.4 ml/min, 0.45 ml/min, 0.50 ml/min, 0.55 ml/min, 0.6 ml/min, 0.65 ml/min, 0.7 ml/min, 0.75 ml/min, 0.8 ml/min, 0.85 ml/min, 0.9 ml/min, 0.95 ml/min, 1.0 ml/min, 1.1 ml/min, 1.2 ml/min, 1.3 ml/min, 1.4 ml/min, 1.5 ml/min, or more, or any range bracketed by any of two values provided.

In certain embodiments (e.g., for a 96 hr continuous infusion), the loading dose rate (the first rate) for coenzyme Q10 administration is at least 1 mg/kg/hr, at least 2 mg/kg/hr, at least 3 mg/kg/hr, at least 4 mg/kg/hr, at least 5 mg/kg/hr, at least 6 mg/kg/hr, at least 7 mg/kg/hr, at least 8 mg/kg/hr, at least 9 mg/kg/hr, at least 10 mg/kg/hr, at least 11 mg/kg/hr, at least 12 mg/kg/hr, at least 13 mg/kg/hr, at least 14 mg/kg/hr, at least 15 mg/kg/hr, at least 16 mg/kg/hr, at least 17 mg/kg/hr, at least 18 mg/kg/hr, at least 19 mg/kg/hr, at least 20 mg/kg/hr, at least 25 mg/kg/hr, at least 30 mg/kg/hr, at least 35 mg/kg/hr, at least 40 mg/kg/hr, at least 45 mg/kg/hr, or at least 50 mg/kg/hr, where “kg” is the body weight of the subject being treated. In particular embodiments, the loading dose rate for coenzyme Q10 administration is about 3.1 mg/kg/hr, about 4.2 mg/kg/hr, about 5.5 mg/kg/hr, about 7.4 mg/kg/hr, about 9.2 mg/kg/hr, about 11 mg/kg/hr, about 14 mg/kg/hr, or about 18 mg/kg/hr. In particular embodiments (e.g., for a 144 hr continuous infusion), the loading dose rate for coenzyme Q10 administration is about 3.1 mg/kg/hr, about 4.1 mg/kg/hr, about 5.4 mg/kg/hr, about 7.2 mg/kg/hr, about 9.0 mg/kg/hr, about 11.2 mg/kg/hr, about 14.0 mg/kg/hr, or about 17.6 mg/kg/hr. Any of these values may be used to define a range for the loading dose rate for coenzyme Q10 administration. For example, the loading dose rate (first rate) may range from about 1 mg/kg/hr to about 50 mg/kg/hr, from about 1 mg/kg/hr to about 20 mg/kg/hr, from about 3 mg/kg/hr to about 15 mg/kg/hr, from about 5 mg/kg/hr to about 20 mg/kg/hr, or from about 3.1 mg/kg/hr to about 18 mg/kg/hr.

In a preferred embodiment, the dose rate for the loading dose (first rate) does not exceed 26.0 mg/kg/hr. In a preferred embodiment, the dose rate does not exceed 19.6 mg/kg/hr. In a preferred embodiment, the dose rate does not exceed 14.5 mg/kg/hr.

In certain embodiments, the rate of the infusion for the remaining 23 hours of the first 24 hours of the dose, the remaining 47 hours of the first 48 hours of the dose, or the remaining 71 hours of the first 72 hours of the dose is about 0.02 ml/min to about 0.4 ml/min. In certain embodiments, the rate of infusion is about 0.02 ml/min, 0.03 ml/min, 0.04 ml/min, 0.05 ml/min, 0.06 ml/min, 0.07 ml/min, 0.08 ml/min, 0.09 ml/min, 0.1 ml/min, 0.11 ml/min, 0.12 ml/min, 0.13 ml/min, 0.14 ml/min, 0.15 ml/min, 0.16 ml/min, 0.17 ml/min, 0.18 ml/min, 0.19 ml/min, 0.20 ml/min, 0.21 ml/min, 0.22 ml/min, 0.23 ml/min, 0.24 ml/min, 0.25 ml/min, 0.30 ml/min, 0.35 ml/min, or more, or any range bracketed by any of two values provided.

In certain embodiments, the dose rate for the remaining 23 hours of a first 24 hour infusion, the remaining 47 hours of a first 48 hour infusion, or the remaining 71 hours of a first 72 hour infusion (i.e. the second dose rate) is at least 0.1 mg/kg/hr, at least 0.2 mg/kg/hr, at least 0.3 mg/kg/hr, at least 0.4 mg/kg/hr, at least 0.5 mg/kg/hr, at least 0.6 mg/kg/hr, at least 0.7 mg/kg/hr, at least 0.8 mg/kg/hr, at least 0.9 mg/kg/hr, at least 1.0 mg/kg/hr, at least 1.5 mg/kg/hr, at least 2.0 mg/kg/hr, at least 2.5 mg/kg/hr, at least 3.0 mg/kg/hr, at least 3.5 mg/kg/hr, at least 4.0 mg/kg/hr, at least 4.5 mg/kg/hr, at least 5.0 mg/kg/hr, at least 6.0 mg/kg/hr, at least 7.0 mg/kg/her, at least 8.0 mg/kg/hr, at least 9.0 mg/kg/hr or at least 10.0 mg/kg/hr. In particular embodiments, the second rate (e.g., for a 48 hour infusion of coenzyme Q10) is about 3.1 mg/kg/hr, about 4.2 mg/kg/hr, about 5.5 mg/kg/hr, about 7.4 mg/kg/hr, about 9.2 mg/kg/hr, about 11 mg/kg/hr, about 14 mg/kg/hr, or about 18 mg/kg/hr. In particular embodiments, the second rate (e.g., for a 72 hour infusion of coenzyme Q10) is about 0.49 mg/kg/hr, about 0.65 mg/kg/hr, about 0.85 mg/kg/hr, about 1.14 mg/kg/hr, about 1.42 mg/kg/hr, about 1.77 mg/kg/hr, about 2.21 mg/kg/hr, or about 2.78 mg/kg/hr. Any of these values may be used to define a range for the second dose rate for coenzyme Q10 administration. For example, the second dose rate may range from about 0.1 mg/kg/hr to about 10 mg/kg/hr, from about 3.1 mg/kg/hr to about 18 mg/kg/hr, from about 0.49 mg/kg/hr to about 2.78 mg/kg/hr, from about 0.5 mg/kg/hr to about 2.5 mg/kg/hr, from about 0.1 mg/kg/hr to about 5 mg/kg/hr, from about 0.1 mg/kg/hr to about 3 mg/kg/hr, or from about 0.5 mg/kg/hr to about 2.2 mg/kg/hr.

In certain embodiments, the second dose rate does not exceed 35.8 mg/kg/hr. In certain embodiments, the second dose rate does not exceed 28.7 mg/kg/hr. In certain embodiments, the second dose rate does not exceed 26 mg/kg/hr. In certain embodiments, the second dose rate does not exceed 22.9 mg/kg/hr. In certain embodiments, the second dose rate does not exceed 19.6 mg/kg/hr. In certain embodiments, the second dose rate does not exceed 18.4 mg/kg/hr. In certain embodiments, the second dose rate does not exceed 14.5 mg/kg/hr. In certain embodiments, the second dose rate does not exceed 11 mg/kg/hr. In certain embodiments, the second dose rate does not exceed 9.2 mg/kg/hr. In certain embodiments, the second dose rate does not exceed 8.3 mg/kg/hr. In certain embodiments, the second dose rate does not exceed 7.4 mg/kg/hr. In certain embodiments, the second dose rate does not exceed 5.5 mg/kg/hr. In certain embodiments, the second dose rate does not exceed 4.2 mg/kg/hr.

Coenzyme Q10 may also be administered at a third rate, i.e. the rate for the second infusion following a short break (e.g. the second 24 hour infusion of a 48 hour infusion, the second 48 hour infusion of a 96 hour continuous infusion, or the second 72 hour infusion of a 144 hour continuous infusion). In certain embodiments, the third rate of infusion is about 0.03 ml/min to about 0.4 ml/min. In certain embodiments, the third rate of infusion is about 0.03 ml/min, 0.04 ml/min, 0.05 ml/min, 0.06 ml/min, 0.07 ml/min, 0.08 ml/min, 0.09 ml/min, 0.1 ml/min, 0.11 ml/min, 0.12 ml/min, 0.13 ml/min, 0.14 ml/min, 0.15 ml/min, 0.16 ml/min, 0.17 ml/min, 0.18 ml/min, 0.19 ml/min, 0.20 ml/min, 0.21 ml/min, 0.22 ml/min, 0.23 ml/min, 0.24 ml/min, 0.25 ml/min, 0.30 ml/min, 0.35 ml/min, or more, or any range bracketed by any of two values provided.

In certain embodiments the third dose rate is at least 0.1 mg/kg/hr, at least 0.2 mg/kg/hr, at least 0.3 mg/kg/hr, at least 0.4 mg/kg/hr, at least 0.5 mg/kg/hr, at least 0.6 mg/kg/hr, at least 0.7 mg/kg/hr, at least 0.8 mg/kg/hr, at least 0.9 mg/kg/hr, at least 1.0 mg/kg/hr, at least 1.5 mg/kg/hr, at least 2.0 mg/kg/hr, at least 2.5 mg/kg/hr, at least 3.0 mg/kg/hr, at least 3.5 mg/kg/hr, at least 4.0 mg/kg/hr, at least 4.5 mg/kg/hr, at least 5.0 mg/kg/hr, at least 6.0 mg/kg/hr, at least 7.0 mg/kg/her, at least 8.0 mg/kg/hr, at least 9.0 mg/kg/hr or at least 10.0 mg/kg/hr. In particular embodiments, the third dose rate (e.g., for a second 48 hour dose of a 96 hour continuous infusion) is about 0.80 mg/kg/hr, about 1.05 mg/kg/hr, about 1.38 mg/kg/hr, about 1.83 mg/kg/hr, about 2.29 mg/kg/hr, about 2.85 mg/kg/hr, about 3.56 mg/kg/hr, or about 4.48 mg/kg/hr. In particular embodiments, the third dose rate (e.g., for a second 72 hour dose of a 144 hour continuous infusion) is about 0.53, about 0.69 mg/kg/hr, about 0.92 mg/kg/hr, about 1.22 mg/kg/hr, about 1.53 mg/kg/hr, about 1.90 mg/kg/hr, about 2.38 mg/kg/hr, or about 2.99 mg/kg/hr. Any of these values may be used to define a range for the third dose rate. For example, the third dose rate may range from about 0.1 mg/kg/hr to about 10 mg/kg/hr, from about 0.80 mg/kg/hr to about 4.48 mg/kg/hr, or from about 0.53 mg/kg/hr to about 2.99 mg/kg/hr.

In certain embodiments, the third dose rate does not exceed 35.8 mg/kg/hr. In certain embodiments, the third dose rate does not exceed 28.7 mg/kg/hr. In certain embodiments, the third dose rate does not exceed 26 mg/kg/hr. In certain embodiments, the third dose rate does not exceed 22.9 mg/kg/hr. In certain embodiments, the third dose rate does not exceed 19.6 mg/kg/hr. In certain embodiments, the third dose rate does not exceed 18.4 mg/kg/hr. In certain embodiments, the third dose rate does not exceed 14.5 mg/kg/hr. In certain embodiments, the third dose rate does not exceed 11 mg/kg/hr. In certain embodiments, the third dose rate does not exceed 9.2 mg/kg/hr. In certain embodiments, the third dose rate does not exceed 8.3 mg/kg/hr. In certain embodiments, the third dose rate does not exceed 7.4 mg/kg/hr. In certain embodiments, the third dose rate does not exceed 5.5 mg/kg/hr. In certain embodiments, the third dose rate does not exceed 4.2 mg/kg/hr.

Coenzyme Q10 may be administered at different daily doses depending on the dosing regimen. For example, in certain embodiments, the daily dose of coenzyme Q10 for the first day of infusion (i.e. the first 24 hours of infusion) is higher than the daily dose for the second day of infusion (i.e. the second 24 hours of infusion) if a loading dose is used. In certain embodiments, the daily dose for the third day of infusion (e.g. hours 49-72 of a 72-hour infusion) is lower than the daily dose for the first day of infusion, and higher than the daily dose for the second day of infusion. In certain embodiments, coenzyme Q10 is administered at a dose of at least 1 mg/kg/day (24 hours), at least 5 mg/kg/day (24 hours), at least 10 mg/kg/day (24 hours), at least 15 mg/kg/day (24 hours), at least 20 mg/kg/day (24 hours), at least 25 mg/kg/day (24 hours), at least 30 mg/kg/day (24 hours), at least 35 mg/kg/day (24 hours), at least 40 mg/kg/day (24 hours), at least 45 mg/kg/day (24 hours), at least 50 mg/kg/day (24 hours), at least 55 mg/kg/day (24 hours), at least 60 mg/kg/day (24 hours), at least 65 mg/kg/day (24 hours), at least 70 mg/kg/day (24 hours), at least 75 mg/kg/day (24 hours), at least 80 mg/kg/day (24 hours), at least 85 mg/kg/day (24 hours), at least 90 mg/kg/day (24 hours), at least 95 mg/kg/day (24 hours), at least 100 mg/kg/day (24 hours), at least 110 mg/kg/day (24 hours), at least 120 mg/kg/day (24 hours), at least 130 mg/kg/day (24 hours), at least 140 mg/kg/day (24 hours), at least 150 mg/kg/day (24 hours), at least 200 mg/kg/day (24 hours), at least 250 mg/kg/day (24 hours), at least 300 mg/kg/day (24 hours), at least 400 mg/kg/day (24 hours), or at least 500 mg/kg/day (24 hours). In particular embodiments, coenzyme Q10 is administered at least one dose selected from the group consisting of about 11.8 mg/kg/day (24 hours), about 12.5 mg/kg/day (24 hours), about 14.4 mg/kg/day (24 hours), about 15.6 mg/kg (24 hours), about 16.5 mg/kg/day (24 hours), about 19 mg/kg/day (24 hours), about 20.4 mg/kg/day (24 hours), about 22 mg/kg/day (24 hours), about 25 mg/kg/day (24 hours), about 27.5 mg/kg/day (24 hours), about 29.3 mg/kg/day (24 hours), about 33 mg/kg/day (24 hours), about 34.2 mg/kg/day (24 hours), about 36.7 mg/kg/day (24 hours), about 41.7 mg/kg/day (24 hours), 42.8 mg/kg/day (24 hours), about 44 mg/kg/day (24 hours), about 45.7 mg/kg/day (24 hours), about 51.9 mg/kg/day (24 hours), about 53.8 mg/kg/day (24 hours), about 55 mg/kg/day (24 hours), about 57 mg/kg/day (24 hours), about 58.7 mg/kg/day (24 hours), about 64.8 mg/kg/day (24 hours), about 66.7 mg/kg/day (24 hours), about 68.5 mg/kg/day (24 hours), about 71.7 mg/kg/day (24 hours), about 73.4 mg/kg/day (24 hours), about 81.5 mg/kg/day (24 hours), about 85.5 mg/kg/day (24 hours), about 91.7 mg/kg/day (24 hours), about 107.5 mg/kg/day (24 hours), about 114.6 mg/kg/day (24 hours), and about 143.3 mg/kg/day (24 hours). Any of these values may be used to define a range for the daily dose of coenzyme Q10. For example, the daily dose of coenzyme Q10 may range from 1 mg/kg/day (24 hours) to 500 mg/kg/day (24 hours), from 10 mg/kg/day (24 hours) to 150 mg/kg/day (24 hours), or from 11.8 mg/kg/day (24 hours) to 143.2 mg/kg/day (24 hours).

In one embodiment, the daily dose (e.g., average daily dose) of coenzyme Q10 for a continuous infusion regimen of the invention (e.g., a 48 hour, 72 hour, 96 hour or 144 hour continuous infusion) ranges from 10-65 mg/kg per day (24 hours), from 10-15 mg/kg per day (24 hours), from 15-20 mg/kg per day (24 hours), from 20-25 mg/kg per day (24 hours), from 25-35 mg/kg per day (24 hours), from 35-42 mg/kg per day (24 hours), from 42-52 mg/kg per day (24 hours), or from 52-65 mg/kg per day (24 hours).

In certain embodiments, the formulation, preferably a coenzyme Q10 formulation, can be administered in one or more cycles. For example, the coenzyme Q10 can be administered for 2, 3, 4, 5, 6, 7, 8, or more weeks consecutively, and then not administered for a period of 1, 2, 3, 4, or more weeks, providing a cycle of administration. In certain embodiments, the cycles are administered without a pause between cycles. In certain embodiments, at the end of one or more cycles, the patient is assessed to determine treatment efficacy, toxicity, and assess if the treatment should be continued, modified, or ended. In certain embodiments, one or more cycles of coenzyme Q10 treatment are administered with one or more cycles of at least one anticancer agent. In certain embodiments, treatment with the additional anti-cancer agent is initiated after the first cycle of treatment with coenzyme Q10. In certain embodiments, treatment with the additional anti-cancer agent is initiated after at least one cycle of treatment with coenzyme Q10. In certain embodiments, treatment with the additional anti-cancer agent is initiated after cancer progression in the subject after treatment with coenzyme Q10. In certain embodiments, treatment with the additional anti-cancer agent is initiated without cancer progression in the subject after treatment with coenzyme Q10.

The number of cycles of administration depends, for example, on the response of the subject, the severity of disease, other therapeutic interventions used on the subject, or any adverse response of the subject. In certain embodiments, the coenzyme Q10 formulation is administered as long as the subject is exhibiting at least a stable response to treatment with no serious adverse events, e.g., dose limiting toxicities, grade IV toxicities, or persistent grade III toxicities that cannot be mitigated by the use of other interventions.

In certain embodiments, the coenzyme Q10 is administered for at least 1, 2, 3, 4, 5, 6, 7, 8, 9, 10, 11, 12, 13, 14, 15, 16, 17, 18, 19, or 20 cycles. In certain embodiments, the coenzyme Q10 is administered for no more than 1, 2, 3, 4, 5, 6, 7, 8, 9, 10, 11, 12, 13, 14, 15, 16, 17, 18, 19, or 20 cycles. Any of these values may be used to define a range for the number of cycles. For example, the number of cycles may range from 1-20, from 2-10, or from 4-8.

In another embodiment, the formulation, preferably, a coenzyme Q10 formulation, is administered in the form of a coenzyme Q10 IV formulation at a dosage of between about 10 mg/kg/dose and about 10,000 mg/kg/dose of coenzyme Q10, about 20 mg/kg/dose to about 5000 mg/kg/dose, about 50 mg/kg/dose to about 3000 mg/kg/dose, about 100 mg/kg/dose to about 2000 mg/kg/dose, about 200 mg/kg/dose to about 1000 mg/kg/dose, about 300 mg/kg/dose to about 500 mg/kg/dose, or about 55 mg/kg/dose to about 110 mg/kg/dose wherein the coenzyme Q10 formulation comprises between about 1% and 10% of coenzyme Q10 (w/v). In one embodiment, the coenzyme Q10 formulation comprises about 4% of coenzyme Q10 (w/v). In one embodiment, the coenzyme Q10 IV formulation comprises about 8% of coenzyme Q10 (w/v). In other embodiments, the coenzyme Q10 IV formulation comprises about 0.1%, 0.2%. 0.3%, 0.4%. 0.5%, 0.6%, 0.7%, 0.8%. 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% of coenzyme Q10 (w/v). It should be understood that ranges having any one of these values as the upper or lower limits are also intended to be part of this invention.

Without wishing to be bound by mechanism, in certain embodiments, the effect coenzyme Q10 may have on cancer cells may depend, in part, on the various states of metabolic and oxidative flux exhibited by the cancer cells. Coenzyme Q10 may be utilized to interrupt and/or interfere with the conversion of an oncogenic cell's dependency of glycolysis and increased lactate utility. As it relates to a cancer state, this interference with the glycolytic and oxidative flux of the tumor microenvironment may influence apoptosis and angiogenesis in a manner which reduces the viability or proliferative capacity of a cancer cell. In some embodiments, the interaction of coenzyme Q10 with glycolytic and oxidative flux factors may enhance the ability of coenzyme Q10 to exert its restorative apoptotic effect in cancer.

In one embodiment, administration of coenzyme Q10 as described herein, achieves at least stable disease, reduces tumor size, inhibits tumor growth and/or prolongs the survival time of a pancreatic tumor-bearing subject as compared to an appropriate control. Accordingly, this invention also relates to a method of treating pancreatic tumors in a human or other animal, including a subject, who has failed at least one prior chemotherapeutic regimen, by administering to such human or animal an effective, non-toxic amount of coenzyme Q10, for example, by administering an effective dose by IV administration, preferably continuous IV administration. One skilled in the art would be able, by routine experimentation with the guidance provided herein, to determine what an effective, non-toxic amount of coenzyme Q10 for continuous IV administration would be for the purpose of treating pancreatic cancer including in a subject who has failed at least one prior chemotherapeutic regimen. For example, a therapeutically active amount of coenzyme Q10 may vary according to factors such as the disease stage (e.g., stage I versus stage IV), age, sex, medical complications (e.g., immunosuppressed conditions or diseases, coagulopathies) and weight of the subject, and the ability of the coenzyme Q10 to elicit a desired response in the subject. The dosage regimen may be adjusted to provide the optimum therapeutic response. For example, several divided doses may be administered daily, the dose may be administered by continuous infusion, or the dose may be proportionally reduced as indicated by the exigencies of the therapeutic situation.

In certain embodiments of the invention, the methods further include a treatment regimen which includes any one of or a combination of surgery, radiation, chemotherapy, e.g., hormone therapy, antibody therapy, therapy with growth factors, cytokines, and anti-angiogenic therapy.

In certain embodiments of the invention, the methods further comprise monitoring the subject for decreased coagulation. In certain embodiments, the subject is monitored for decreased coagulation before administering coenzyme Q10 to the subject. In certain embodiments, the subject is monitored for decreased coagulation after administering coenzyme Q10 to the subject. In certain embodiments, the subject is monitored for decreased coagulation before administering coenzyme Q10 to the subject and after administering coenzyme Q10 to the subject. In certain embodiments, monitoring for decreased coagulation comprises assessing the PT/PTT, INR and/or platelet count. In certain embodiments, decreased coagulation comprises an INR of greater than 2 and normal coagulation comprises an INR of 2 or less. In certain embodiments, the decreased coagulation comprises an INR of greater than 3 and normal coagulation comprises an INR of 3 or less. In certain embodiments, decreased coagulation comprises a platelet threshold less than 50,000/μL. In certain embodiments, normal coagulation comprises a platelet threshold of at least 50,000/μL.

In certain embodiments, the methods further comprise administering an agent to increase coagulation in a subject identified as having decreased coagulation. In certain embodiments, the agent to increase coagulation is administered before administering coenzyme Q10 to the subject. In certain embodiments, the agent to increase coagulation is administered after administering coenzyme Q10 to the subject. In a particular embodiment, the agent to increase coagulation comprises vitamin K. In certain embodiments, the vitamin K is administered by oral, intravenous, intramuscular, or subcutaneous administration. In a particular embodiment, the agent to increase coagulation comprises cryoprecipitate or fresh frozen plasma.

In certain embodiments, the methods further comprise discontinuing treatment with coenzyme Q10 in a subject identified as having decreased coagulation. In certain embodiments, the methods further comprise confirming the subject has normal coagulation and starting treatment with coenzyme Q10. In certain embodiments, coenzyme Q10 is administered to the subject if the INR, PT, and PTT are less than or equal to 1.5 times the upper limit of normal and the platelet count is greater than or equal to 50,000/μL. In certain embodiments, the methods further comprise confirming the subject has normal coagulation and continuing treatment with coenzyme Q10. In certain embodiments, the coenzyme Q10 is administered at two, three, four or five different rates. In certain embodiments, the coenzyme Q10 is administered at two or more different rates, three or more different rates, four or more different rates, or five or more different rates. In certain embodiments, the coenzyme Q10 is administered for at least 24 hours, at least 48 hours, at least 72 hours, at least 96 hours, at least 120 hours, at least 144 hours, at least 168 hours, at least 192 hours, at least 216 hours, at least 240 hours, at least 264 hours, at least 288 hours, at least 312 hours, at least 336 hours, at least 360 hours, at least 384 hours, at least 408 hours, at least 432 hours, at least 456 hours, or at least 480 hours.

### VI. Combination Therapies

In certain embodiments, the formulations and methods of the invention can be used in combination therapy with at least one additional anticancer agent, e.g., chemotherapeutic agent. In certain embodiments, treatment with the chemotherapeutic agent is initiated at the same time as treatment with the coenzyme Q10. In certain embodiments, the treatment with the chemotherapeutic agent is initiated after the treatment with coenzyme Q10 is initiated. In certain embodiments, treatment with the additional agent is initiated upon progression of the pancreatic cancer during treatment with coenzyme Q10. In certain embodiments, treatment with the additional agent is initiated without progression of the pancreatic cancer during treatment with coenzyme Q10. In certain embodiments, treatment with coenzyme Q10 is continued upon initiation of administration of the additional agent. In certain embodiments, treatment with coenzyme Q10 is stopped upon initiation of treatment with the additional agent.

The methods of treatment of cancer by continuous infusion of coenzyme Q10 provided herein include combination therapies with additional anticancer agents or interventions (e.g., radiation, surgery, bone marrow transplant). In certain embodiments, “combination therapy” includes a treatment with coenzyme Q10 to decrease tumor burden and/or improve clinical response. Administration of coenzyme Q10 with palliative treatments or treatments to mitigate drug side effects (e.g., to decrease nausea, pain, anxiety, or inflammation, to normalize clotting) is not considered to be a combination treatment of the cancer.

In a preferred embodiment, treatment with coenzyme Q10 by continuous infusion is combined with the standard of care for treatment of pancreatic cancer. The standard of care for pancreatic cancer can be determined by one of skill in the art based on, for example, the type and severity of the pancreatic cancer, the age, weight, gender, and/or medical history of the subject, and the success or failure of prior treatments.

In certain embodiments, treatment of subjects with pancreatic cancer by continuous infusion of coenzyme Q10 is combined with one or more of the following treatments.

1. Gemcitabine, preferably by intravenous administration at a weekly dose starting at 600 mg/m², with the dose being adjusted based on the tolerance of the subject to the drug.

2. 5-Fluorouracil (5-FU), preferably by intravenous administration at a weekly starting dose of 350 mg/m², with the dose being adjusted based on the tolerance of the subject to the drug.

In certain embodiments, treatment with gemcitabine and/or 5-FU is initiated after completion of a 144 hour continuous intravenous infusion of coenzyme Q10 alone. In certain embodiments, 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

In certain embodiments, coenzyme Q10 is administered in an amount that would be therapeutically effective if delivered alone, i.e., coenzyme Q10 is administered and/or acts as a therapeutic agent, and not predominantly as an agent to ameliorate side effects of other chemotherapy or other cancer treatments. Coenzyme Q10 and/or pharmaceutical formulations thereof and the other therapeutic agent can act additively or, more preferably, synergistically. In one embodiment, coenzyme Q10 and/or a formulation thereof is administered concurrently with the administration of an additional anticancer (e.g., chemotherapeutic, anti-angiogenic) agent. In another embodiment, a compound and/or pharmaceutical formulation thereof is administered prior or subsequent to administration of another anticancer agent wherein both agents are present in the subject at the same time or have therapeutic activity in the subject at the same time. In one embodiment, the coenzyme Q10 and additional anticancer agent act synergistically. In one embodiment, the coenzyme Q10 and additional anticancer agent act additively.

In one embodiment, the therapeutic methods of the invention further comprise administration of one or more additional therapeutic agents, e.g., one or more anticancer agents, e.g., anti-angiogenic agents, chemotherapeutic agents, e.g., small molecule anticancer agents, biologic anticancer agents including both protein based and nucleic acid based therapeutics. For example, in one embodiment, an additional anticancer agent for use in the therapeutic methods of the invention is a chemotherapeutic agent. In certain embodiments, the chemotherapeutic agent is selected from the group consisting of cisplatin, gemcitabine (gemzar), mitomycin, 5-fluorouracil (5-FU), paclitaxel (taxol), and irinotecan.

In certain aspects, the disclosure relates to a method of treating pancreatic cancer in a population of subjects in need thereof, the method comprising administering to each subject of the population a composition comprising Coenzyme Q10, wherein at least 10%, 20%, 30%, 40%, 50% or 53% of the subjects in the population exhibit stable disease as a result of administration of the Coenzyme Q10 to the subjects, wherein the subjects have failed treatment for the pancreatic cancer with at least one prior therapy, and wherein the method comprises administering to each subject of the population: 1) a composition comprising Coenzyme Q10, wherein the composition is administered to the subject by continuous intravenous infusion for at least 144 hours at a dose of at least 110 mg/kg/72 hours; and 2) gemcitabine, wherein the gemcitabine is administered intravenously at a dose of at least 1000 mg/m², wherein the continuous intravenous infusion of Coenzyme Q10 is administered to the subject for at least 30 days over a ten week period, thereby treating the pancreatic cancer in the population of subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median progression free survival of at least 50, 60, 70, 80, 90, 100, 110, or 118 days as a result of administration of the Coenzyme Q10 to the subjects, wherein the subjects have failed treatment for the pancreatic cancer with at least one prior therapy, and wherein the method comprises administering to each subject of the population: 1) a composition comprising Coenzyme Q10, wherein the composition is administered to the subject by continuous intravenous infusion for at least 144 hours at a dose of at least 110 mg/kg/72 hours; and 2) gemcitabine, wherein the gemcitabine is administered intravenously at a dose of at least 1000 mg/m², wherein the continuous intravenous infusion of Coenzyme Q10 is administered to the subject for at least 30 days over a ten week period, thereby treating the pancreatic cancer in the population of subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median overall survival of at least 50, 60, 70, 80, 90, 100, 110, 120, 125, 150, 175, 200 or 218 days as a result of administration of the Coenzyme Q10 to the subjects, wherein the subjects have failed treatment for the pancreatic cancer with at least one prior therapy, and wherein the method comprises administering to each subject of the population: 1) a composition comprising Coenzyme Q10, wherein the composition is administered to the subject by continuous intravenous infusion for at least 144 hours at a dose of at least 110 mg/kg/72 hours; and 2) gemcitabine, wherein the gemcitabine is administered intravenously at a dose of at least 1000 mg/m², wherein the continuous intravenous infusion of Coenzyme Q10 is administered to the subject for at least 30 days over a ten week period, thereby treating the pancreatic cancer in the population of subjects.

In certain aspects, the disclosure relates to a method of treating pancreatic cancer in a population of subjects in need thereof, the method comprising administering to each subject of the population a composition comprising Coenzyme Q10, wherein the population of subjects exhibits a median time to progression of at least 10, 20, 30, 40, 50, 60, 65, 68, 70, 80, 90, 100, 110, 120 or 121 days as a result of administration of the Coenzyme Q10 to the subjects, wherein the subjects have failed treatment for the pancreatic cancer with at least one prior therapy, and wherein the method comprises administering to each subject of the population: 1) a composition comprising Coenzyme Q10, wherein the composition is administered to the subject by continuous intravenous infusion for at least 144 hours at a dose of at least 110 mg/kg/72 hours; and 2) gemcitabine, wherein the gemcitabine is administered intravenously at a dose of at least 1000 mg/m², wherein the continuous intravenous infusion of Coenzyme Q10 is administered to the subject for at least 30 days over a ten week period, thereby treating the pancreatic cancer in the population of subjects.

In certain embodiments, the methods disclosed herein further comprise selecting for a subject having pancreatic cancer that has had at least one prior therapy for the pancreatic cancer. In certain embodiments, the methods disclosed herein further comprise selecting for a subject having pancreatic cancer that has had no more than 2 prior therapies for the pancreatic cancer. In certain embodiments, the methods disclosed herein further comprise selecting for a subject having pancreatic cancer that has failed treatment for the pancreatic cancer with at least one prior therapy. In certain embodiments, the methods disclosed herein further comprise selecting for a subject having metastatic pancreatic cancer.

In some embodiments, the composition is administered to the subject by continuous intravenous infusion for at least 144 hours at a dose of at least 110 mg/kg/72 hours once per week for at least 2 weeks. In some embodiments, the composition is administered to the subject by continuous intravenous infusion for at least 144 hours at a dose of at least 110 mg/kg/72 hours once per week for at least 3 weeks. In some embodiments, the composition is administered to the subject by continuous intravenous infusion for at least 144 hours at a dose of at least 110 mg/kg/72 hours once per week for at least 4 weeks. In some embodiments, the composition is administered to the subject by continuous intravenous infusion for at least 144 hours at a dose of at least 110 mg/kg/72 hours once per week for at least 5 weeks. In some embodiments, the composition is administered to the subject by continuous intravenous infusion for at least 144 hours at a dose of at least 110 mg/kg/72 hours once per week for at least 6 weeks. In some embodiments, the composition is administered to the subject by continuous intravenous infusion for at least 144 hours at a dose of at least 110 mg/kg/72 hours once per week for at least 7 weeks. In some embodiments, the composition is administered to the subject by continuous intravenous infusion for at least 144 hours at a dose of at least 110 mg/kg/72 hours once per week for at least 8 weeks. In some embodiments, the composition is administered to the subject by continuous intravenous infusion for at least 144 hours at a dose of at least 110 mg/kg/72 hours once per week for at least 9 weeks. In some embodiments, the composition is administered to the subject by continuous intravenous infusion for at least 144 hours at a dose of at least 110 mg/kg/72 hours once per week for at least 10 weeks.

In some embodiments, the gemcitabine is administered intravenously at a dose of at least 1000 mg/m² once per week for at least 2 weeks. In some embodiments, the gemcitabine is administered intravenously at a dose of at least 1000 mg/m² once per week for at least 3 weeks. In some embodiments, the gemcitabine is administered intravenously at a dose of at least 1000 mg/m² once per week for at least 4 weeks. In some embodiments, the gemcitabine is administered intravenously at a dose of at least 1000 mg/m² once per week for at least 5 weeks. In some embodiments, the gemcitabine is administered intravenously at a dose of at least 1000 mg/m² once per week for at least 6 weeks.

### EXAMPLES

### Example 1—Phase 2 Clinical Investigation of Coenzyme Q10 Alone and in Combination with Gemcitabine in Patients with Advanced Pancreatic Cancer

The composition comprising Coenzyme Q10 evaluated in this study is a ubidecarenone containing intravenous (IV) nanodispersion that switches cancer energy generation from glycolysis to mitochondrial oxidative phosphorylation to elicit an anticancer effect. This Coenzyme Q10 IV composition was demonstrated to be well tolerated as a monotherapy and in combination with gemcitabine with established MTD of 110 mg/kg in a Phase I clinical trial. Preclinical in vivo pancreatic models demonstrated that IV administration of Coenzyme Q10 alone and in combination with gemcitabine significantly improved duration of survival outcomes; providing support for this Phase 2 evaluation of IV formulations of Coenzyme Q10 in patients with advanced metastatic adenocarcinoma.

Eligible patients (aged ≥18 y) were relapsed/refractory to standard treatment and met inclusion/exclusion criteria. Each patient had metastatic pancreatic cancer and had at least one, prior, but no more than 2, prior standard or experimental therapies. Each received either two 72-hr infusions of 110 mg/kg IV Coenzyme Q10 alone (Monotherapy) or in combination with gemcitabine (Combination Therapy), per week. Tumor response was evaluated at week 10 and then every 8 weeks. Clinical assessments included investigator observations and patient reports. This study occurred in two parts: Part 1 of the study enrolled ten (10) patients in the Coenzyme Q10 (monotherapy arm) and ten (10) patients in the Coenzyme Q10 plus gemcitabine (combination therapy arm) with intent to enroll the additional 15 patients (Part 2) into the applicable treatment arm(s) based on RECIST v1.1 clinical response.

Patients with no prior gemcitabine treatments prior to screening for the study, or prior gemcitabine treatment >3 months prior to screening for the study were assigned to the combination therapy group. Patients with prior gemcitabine discontinued due to toxicity in prior treatment, patients unable to tolerate a dose of 800 mg/m² weekly at any point due to toxicity, and patients with prior gemcitabine treatment ≤3 months prior to screening for the study were assigned to the monotherapy group.

A Coenzyme Q10 Nanosuspension Injection (40 mg/mL) was administered IV over 144 hours. Each patient received 2 consecutive 72-hour infusions per week. Monotherapy arm patients received a Coenzyme Q10 dose of 110 mg/Kg only weekly. Patients in the combination therapy arm received a Coenzyme Q10 dose of 110 mg/Kg and gemcitabine IV once weekly (starting at week 3) at a starting dose of 1000 mg/m².

The primary objective of the study was to evaluate the Overall Response Rate (ORR) in patients treated with the combination of Coenzyme Q10 with gemcitabine or Coenzyme Q10 as a monotherapy. The secondary objectives were to evaluate Overall Survival (OS), Progression-Free Survival (PFS) overall and at around 10 weeks (equal to 2 cycles of treatment), Time to Progression (TTP), Tumor Response using Adaptive Molecular Responses (multi-omic molecular profiling), the toxicity profile of Coenzyme Q10 with gemcitabine and as monotherapy when given over 144-hour intravenous (IV) infusion in patients with advanced pancreatic cancer, changes in carbohydrate antigen (CA) 19-9 levels, and patient reported Quality of Life (QOL) using the validated FACT-HEP PRO. A comprehensive multi-omic profiling for identification of biomarkers for patient stratification was also explored. The exploratory objectives were to evaluate the effects of Coenzyme Q10 on shifting tumors to aerobic respiration by 18-FDG-PET imaging. Tumor, plasma and urine samples were assayed for levels of markers of Coenzyme Q10 activity using (but not limited to): genomics, proteomics, metabolomics and lipidomics with the intent of stratifying patient populations based on phenotypic or molecular profiles for therapeutic benefit.

### Key Eligibility Criteria

The patient has a histologically or cytologically confirmed metastatic pancreatic adenocarcinoma.

The patient has received: at least one, prior, but no more than 2, prior standard or experimental therapies for metastatic pancreatic cancer. If the patient has had prior gemcitabine treatment, the last date of gemcitabine administration-should be >3 months prior to screening for the study. All patients who have previously received gemcitabine should be discussed with the medical monitor during screening.

The patient has adequate coagulation.

The patient has an ECOG performance status 0-1.

Measurable tumor lesions according to RECIST 1.1.

Patients previously treated with gemcitabine: who discontinued gemcitabine due to toxicity in the first 6 weeks of prior treatment or is/was unable to tolerate a dose of 800 mg/m2 weekly at any point due to toxicity are not eligible for combination arm.

### Demographics

A total of 47 patients consented, 13 evaluable, 4 were active. Overall, 31 patients received at least 1 treatment in this study. 37% of patients completed the study.

TABLE 1
Overview of pancreatic cancer patients.

| Overall to Date (N = 47) | N(%) |
|---|---|
| Number of Patients Consented | 47 |
| Patients Treated, n (%) | 31 (66.0) |
| Completed, n (%) | 13 (27.6) |
| Discontinued, n (%) | 18 (38.2) |
| Reasons for discontinuation from the study | N (%) |
| Adverse events | 4 (0.9) |
| Non-compliance | 0 |
| Protocol Violation | 0 |
| Disease Progression | 12 (25.5) |
| Other | 2 (0.4) |

TABLE 2
Characteristics of pancreatic
cancer patients

| Factor | N (%) |
|---|---|
| Age (years) | |
| N | 31 |
| Mean | 63 |
| Median | 63 |
| Minimum, Maximum | 39,80 |
| Sex, n (%) | |
| Male | 18 (58.1) |
| Female | 13 (41.9) |
| Ethnicity, n (%) | |
| Hispanic/Latino | 2 (6.4) |
| Non-Hispanic/Latino | 24 (77.4) |
| Unknown/Not Reported | 5 (16.2) |
| Race, n (%) | |
| White | 30 (96.7) |
| Black, African American | 1 (3.3) |
| Asian | 0 |
| Other | 0 |

### Results

### Efficacy

A preliminary review of efficacy data shows:

Early indication of clinical response in reversal of metabolism
Some patients demonstrated pseudoprogression
7 stable disease (SD) noted (1 monotherapy and 6 combination therapy)/13 evaluable patients
3 patients have SD >4 months
4 patients have SD<4 months

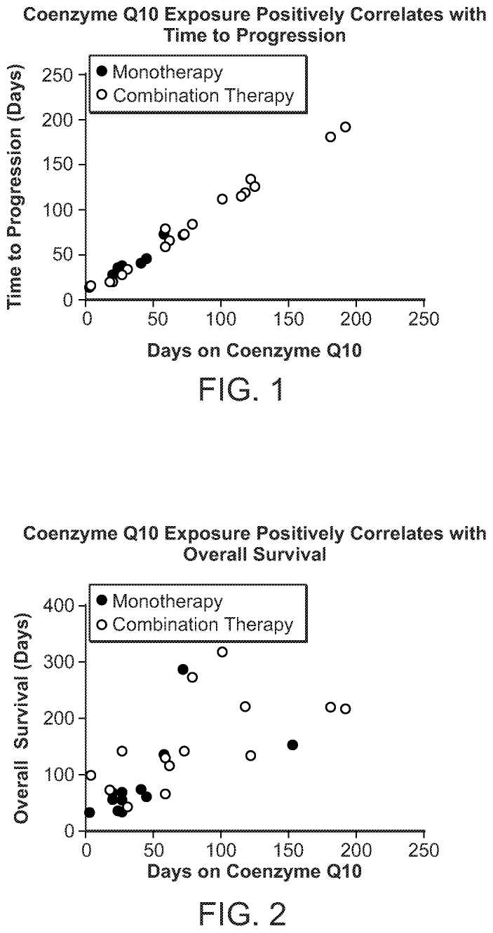

As shown in FIG. 1, time of Coenzyme Q10 exposure was positively correlated with time to progression. As shown in FIG. 2, time of Coenzyme Q10 exposure was also positively correlated with overall survival.

### Adverse Events (AE)

All patients experienced at least 1 AE
INR increase (48.4%), fatigue (38/7%), abdominal pain (35.5%) and thrombocytopenia (35.5%) were the most frequently experienced AE
10 AEs reported were considered definitely related to study drug
14 AEs reported were considered probably related to study drug
19 AEs reported were considered possibly related to study drug

TABLE 3
Summary of Treatment-Emergent Adverse
Events—Safety Population

| | N (%) |
|---|---|
| Patients with an AE | 31 (100) |
| Mono PTs an AE | 13 (41.9) |
| Combo PTs an AE | 18 (58.1) |
| Number of AEs | 347 |
| Subjects with any: | |
| Serious AE | 12 (38.7) |
| ≥Grade 3 AE | 31 (100) |
| AE leading to study drug discontinuation | 4 (12.9) |
| Infusion reactions | 0 |

Abbreviations: AE, adverse event; Mono, Coenzyme Q10-IV monotherapy; Combo, Coenzyme Q10-IV Plus Gemcitabine Combination Therapy; PTs, patients

### Summary

In the overall current population, mean duration of treatment was 63.7 days, mean overall survival was 125 days (Median 108 days), mean time to progression (TTP) was 68 days (Median 53 days). Coenzyme Q10-IV was well-tolerated. 47 patients consented, 13 evaluable, 4 active (2 in screening).

### Example 2—Further Results of Phase 2 Clinical Investigation of Coenzyme Q10 Alone and in Combination with Gemcitabine in Patients with Advanced Pancreatic Cancer

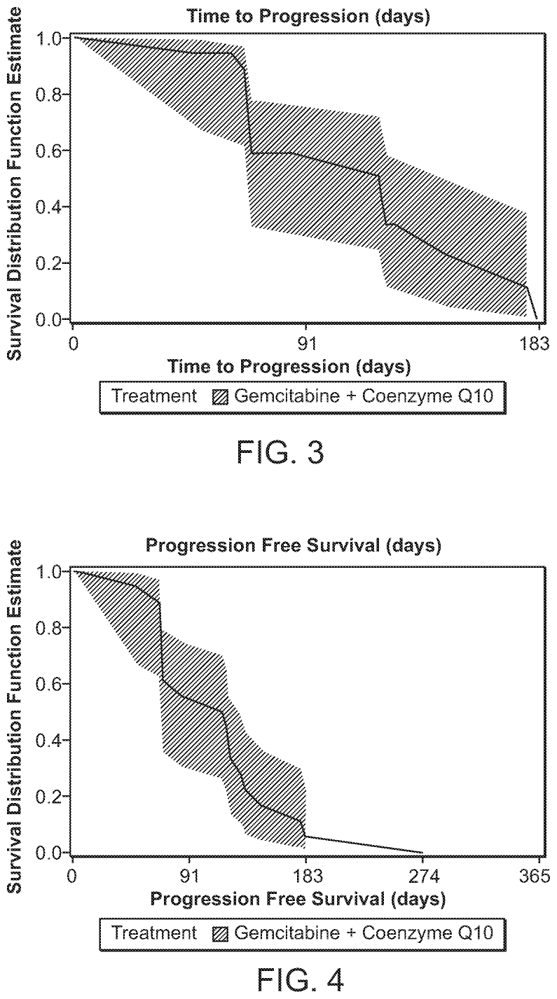

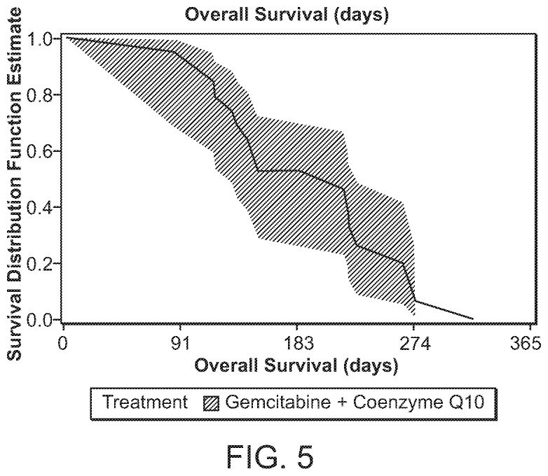

The study described above in Example 1 was continued and additional patients were evaluated. Of the 35 patients enrolled to receive therapy, 18 patients met the criteria of an adequately treated cohort (ATC). Patients in the ATC received Coenzyme Q10 for at least 30 days over a ten week period, at least one dose of gemcitabine, and had a RECIST 1.1 evaluation. The remaining patients (n=17) had progressive disease (PD). Half of the ATC population (n=9/18, 50%) achieved best ORR of stable disease (SD); 10/18 (55%) demonstrated SD as best response at target lesions and 8/18 demonstrated SD at end of Cycle 2. The median Time to Progression (TTP) was 121 days (70-147, 95% CI) (FIG. 3). The median Progression-Free Survival (PFS) was 118 days (70-131, 95% CI) (FIG. 4). The median Overall Survival (OS) was 218 days (131-228, 95% CI) (FIG. 5). Overall, Coenzyme Q10 was well tolerated; the most common AE's were GI related.

TABLE 4
Characteristics of adequately treated cohort (ATC) of pancreatic
cancer patients in study. For Eastern Cooperative Oncology Group
(ECOG) performance status, 0 is asymptomatic and 1 is
symptomatic but completely ambulatory.

| | Gemcitabine + Coenzyme |
|---|---|
| | Q10 |
| Parameters | (N = 18) |
| Age (years) | |
| Median (Range) | 64.5 (38-79) |
| Sex-n (%) | |
| Female | 7 (38.9) |
| Male | 11 (61.1) |
| Eastern Cooperative Oncology Group | |
| (ECOG) Performance Status S-n (%) | |
| 0 | 10 (55.5) |
| 1 | 8 (44.4) |
| No of Prior Therapies-n (%) | |
| 1 | 5 (27.7) |
| ≥2 | 13 (72.2) |

TABLE 5
RECIST Summary: Best Responses
(Adequately Treated Combo Arm Population)

| | Best Overall | Best Target | Best Non-Target |
|---|---|---|---|
| | Response | Lesion Response | Lesion Response |
| Patient | in Study | During Study | (RECIST) |
| 1 | SD | SD | SD |
| 2 | PD | PD | SD |
| 3 | PD | PD | SD |
| 4 | SD | SD | SD |
| 5 | SD | SD | PD |
| 6 | SD | SD | PD |
| 7 | PD | PD | ? |
| 8 | SD | SD | ? |
| 9 | PD | PD | PD |
| 10 | SD | SD | NE |
| 11 | PD | PD | ? |
| 12 | SD | SD | SD |
| 13 | PD | PD | SD |
| 14 | PD | PD | NE |
| 15 | SD | SD | SD |
| 16 | PD | SD | PD |
| 17 | NE | NE | NE |
| 18 | SD | SD | SD |

TABLE 6
RECIST Criteria at each time point: Best Responses
(Adequately Treated Combo Arm Population)

| | | | | End of |
|---|---|---|---|---|
| Patient | Cycle 2 | Cycle 4 | Cycle 6 | Treatment |
| 1 | SD | PD | PD | PD |
| 2 | PD | PD | | PD |
| 3 | PD | | | PD |
| 4 | SD | SD | | SD |
| 5 | SD | SD | PD | |
| 6 | SD | | | PD |
| 7 | PD | | | |
| 8 | SD | | | |
| 9 | PD | PD | | PD |
| 10 | SD | | | |
| 11 | PD | | | PD |
| 12 | | | | SD |
| 13 | PD | | | PD |
| 14 | | | | PD |
| 15 | SD | SD | | PD |
| 16 | | | | PD |
| 17 | NE | | | |
| 18 | SD | | | PD |

1. A method of treating pancreatic cancer in a population of subjects in need thereof, the method comprising administering to each subject of the population a composition comprising Coenzyme Q10, wherein at least 10%, 20%, 30%, 40%, 50% or 53% of the subjects in the population exhibit stable disease as a result of administration of the Coenzyme Q10 to the subjects, thereby treating the pancreatic cancer in the population of subjects.

2. The method of claim 1, wherein at least 5%, 10%, 15%, 20% or 23% of the subjects in the population exhibit stable disease for at least four months as a result of administration of the Coenzyme Q10 to the subjects.

3. A method of treating pancreatic cancer in a population of subjects in need thereof, the method comprising administering to each subject of the population a composition comprising Coenzyme Q10, wherein the population of subjects exhibits a median progression free survival of at least 50, 60, 70, 80, 90, 100, 110, or 118 days as a result of administration of the Coenzyme Q10 to the subjects, thereby treating the pancreatic cancer in the population of subjects.

4. A method of treating pancreatic cancer in a population of subjects in need thereof, the method comprising administering to each subject of the population a composition comprising Coenzyme Q10, wherein the population of subjects exhibits a median overall survival of at least 50, 60, 70, 80, 90, 100, 110, 120, 125, 150, 175, 200 or 218 days as a result of administration of the Coenzyme Q10 to the subjects, thereby treating the pancreatic cancer in the population of subjects.

5. A method of treating pancreatic cancer in a population of subjects in need thereof, the method comprising administering to each subject of the population a composition comprising Coenzyme Q10, wherein the population of subjects exhibits a median time to progression of at least 10, 20, 30, 40, 50, 60, 65, 68, 70, 80, 90, 100, 110, 120 or 121 days as a result of administration of the Coenzyme Q10 to the subjects, thereby treating the pancreatic cancer in the population of subjects.

6. The method of claim 1, wherein the composition comprising Coenzyme Q10 is formulated for intravenous administration.

7. The method of claim 1, wherein the composition comprising Coenzyme Q10 is administered by continuous intravenous infusion.

8. The method of claim 7, wherein the composition comprising Coenzyme Q10 is administered by continuous intravenous infusion for at least 24, 48, 72, 96, 120 or 144 hours.

9. The method of claim 7, wherein the composition comprising Coenzyme Q10 is administered by continuous intravenous infusion for at least 144 hours.

10. The method of claim 9, wherein the continuous intravenous infusion is administered to the subject for at least 30 days over a ten week period.

11. The method of claim 1, wherein the coenzyme Q10 is administered at a dose of at least 20, 30, 40, 50, 60, 70, 80, 90, 100 or 110 mg/kg/72 hours.

12. The method of claim 1, wherein the coenzyme Q10 is administered at a dose of at least 110 mg/kg/72 hours.

13. The method of claim 1, wherein the subject is a human.

14. The method of claim 1, wherein the pancreatic cancer is metastatic.

15. The method of claim 1, wherein each subject in the population has had at least one prior therapy for the pancreatic cancer.

16. The method of claim 1, wherein each subject in the population has had no more than 2 prior therapies for the pancreatic cancer.

17. The method of claim 16, wherein each subject in the population has failed treatment for the pancreatic cancer with at least one prior therapy.

18. The method of claim 1, wherein the composition comprising Coenzyme Q10 is administered with one or more additional agents.

19. The method of claim 18, wherein the additional agent is an anti-cancer agent.

20. The method of claim 18, wherein the additional agent is a chemotherapeutic agent.

21. The method of claim 20, wherein the chemotherapeutic agent is selected from the group consisting of cisplatin, gemcitabine (gemzar), mitomycin, 5-fluorouracil (5-FU), paclitaxel (taxol), and irinotecan.

22. The method of claim 20, wherein the chemotherapeutic agent is gemcitabine.

23. The method of claim 22, wherein the gemcitabine is administered intravenously at a dose of at least 1000 mg/m².

24. The method of claim 1, further comprising evaluating each of the subjects by RECIST criteria.

25. The method of claim 1, wherein each of the subjects is an evaluable subject.
